SEQUENCE LISTING (47 sequences)

SEQ ID NO: 1 - DNA, 65 bp, synthetic construct
  source 1..65: mol_type = other DNA
aaaccatggtacgcgcccggggagcccaagggcacgccctggcacccgtcgcgaactagtaatga

SEQ ID NO: 2 - DNA, 32 bp, synthetic construct
  source 1..32: mol_type = other DNA
atcgacgacgtctaatggtgatggtgatggtg

SEQ ID NO: 3 - DNA, 73 bp, synthetic construct
  source 1..73: mol_type = other DNA
gcgcgctctagactccaaaaaaaaaggctccaaaaggagcctttaattgtatcgacgtctaatggtgatggtg

SEQ ID NO: 4 - DNA, 65 bp, synthetic construct
  source 1..65: mol_type = other DNA
atatatgagctccccaactggggtaacctttgagttctctcagttggggcgctagcttcacgctg

SEQ ID NO: 5 - DNA, 44 bp, synthetic construct
  source 1..44: mol_type = other DNA
atatatgcggccgcaggaggatatacatatggtgagcaagggcg

SEQ ID NO: 6 - DNA, 29 bp, synthetic construct
  source 1..29: mol_type = other DNA
atatatgcggccgcgcgaaacgatcctca

SEQ ID NO: 7 - DNA, 25 bp, synthetic construct
  source 1..25: mol_type = other DNA
atcgccatatggtggacacgtacgc

SEQ ID NO: 8 - DNA, 28 bp, synthetic construct
  source 1..28: mol_type = other DNA
atatatgagctcctacgccgctacgtct

SEQ ID NO: 9 - DNA, 32 bp, synthetic construct
  source 1..32: mol_type = other DNA
atgcgcccatggctgattgacaccatcgaatg

SEQ ID NO: 10 - DNA, 32 bp, synthetic construct
  source 1..32: mol_type = other DNA
gcgcgaacatgtctgattgacaccatcgaatg

SEQ ID NO: 11 - DNA, 27 bp, synthetic construct
  source 1..27: mol_type = other DNA
atatagcatatggtggacacgtacgcg

SEQ ID NO: 12 - DNA, 28 bp, synthetic construct
  source 1..28: mol_type = other DNA
atatatgagctcctacgccgctacgtct

SEQ ID NO: 13 - DNA, 48 bp, synthetic construct
  source 1..48: mol_type = other DNA
agagacatgtaggaggggcgcgccatgattgaacaagatggattgcac

SEQ ID NO: 14 - DNA, 33 bp, synthetic construct
  source 1..33: mol_type = other DNA
agagcatatgtcagaagaactcgtcaagaaggc

SEQ ID NO: 15 - DNA, 53 bp, synthetic construct
  source 1..53: mol_type = other DNA
atcgatacatgtaggaggggcgcgccatggagaaaaaaatcactggatatacc

SEQ ID NO: 16 - DNA, 34 bp, synthetic construct
  source 1..34: mol_type = other DNA
atcgatcatatgttaatgaatcggccaacgcgcg

SEQ ID NO: 17 - DNA, 26 bp, synthetic construct
  source 1..26: mol_type = other DNA
aacagcatatgaacgaagcagtcacc

SEQ ID NO: 18 - DNA, 55 bp, synthetic construct
  source 1..55: mol_type = other DNA
atcgcgtctagagtccaaaaaaaaaggctccaaaaggagcctttaattgtatctc

SEQ ID NO: 19 - DNA, 24 bp, synthetic construct
  source 1..24: mol_type = other DNA
atcgaccatatggaccagtcgaac

SEQ ID NO: 20 - DNA, 55 bp, synthetic construct
  source 1..55: mol_type = other DNA
atcgtctagagtccaaaaaaaaaggctccaaaaggagcctttaattgtatcttac

SEQ ID NO: 21 - DNA, 32 bp, synthetic construct
  source 1..32: mol_type = other DNA
tagttaggatcccagcgaggtttgctgcttag

SEQ ID NO: 22 - DNA, 33 bp, synthetic construct
  source 1..33: mol_type = other DNA
tcatcgctgcagatcgggcccggtatcgctcac

SEQ ID NO: 23 - DNA, 33 bp, synthetic construct
  source 1..33: mol_type = other DNA
tcagtaggatccgtcacccacacacacggagga

SEQ ID NO: 24 - DNA, 37 bp, synthetic construct
  source 1..37: mol_type = other DNA
tacgtaactagttcttgccgagcttctccgcctgctt

SEQ ID NO: 25 - DNA, 23 bp, synthetic construct
  source 1..23: mol_type = other DNA
atggcgaaatcagaccaaccgac

SEQ ID NO: 26 - DNA, 22 bp, synthetic construct
  source 1..22: mol_type = other DNA
ctattcgtccttcttcggctgt

SEQ ID NO: 27 - DNA, 21 bp, synthetic construct
  source 1..21: mol_type = other DNA
gtcaatccggcacacgcatct

SEQ ID NO: 28 - DNA, 21 bp, synthetic construct
  source 1..21: mol_type = other DNA
gcaaagccgtcaattagcaag

SEQ ID NO: 29 - DNA, 22 bp, synthetic construct
  source 1..22: mol_type = other DNA
tcgttgattcccgacgcagaac

SEQ ID NO: 30 - DNA, 24 bp, synthetic construct
  source 1..24: mol_type = other DNA
tcgaagacatccggttctgacatg

SEQ ID NO: 31 - DNA, 19 bp, synthetic construct
  source 1..19: mol_type = other DNA
cttatcctccgtgtgtgtg

SEQ ID NO: 32 - DNA, 20 bp, synthetic construct
  source 1..20: mol_type = other DNA
cggactggtgctgaagaact

SEQ ID NO: 33 - DNA, 35 bp, synthetic construct
  source 1..35: mol_type = other DNA
ctacgtggatccttactacgcgtcgtcgttagctg

SEQ ID NO: 34 - DNA, 33 bp, synthetic construct
  unsure 1..33
  source 1..33: mol_type = other DNA
tgagtactagtaggtcgttgccgtaggagtcga

SEQ ID NO: 35 - DNA, 32 bp, synthetic construct
  source 1..32: mol_type = other DNA
tactctggatcccgtgtcgtgtccctcgtaac

SEQ ID NO: 36 - DNA, 36 bp, synthetic construct
  source 1..36: mol_type = other DNA
gtcatgactagtaggtcgcatctggaaagttatcgc

SEQ ID NO: 37 - DNA, 18 bp, synthetic construct
  source 1..18: mol_type = other DNA
cccaactgcttcaacctg

SEQ ID NO: 38 - DNA, 20 bp, synthetic construct
  source 1..20: mol_type = other DNA
atagccgcagacctcgttct

SEQ ID NO: 39 - DNA, 19 bp, synthetic construct
  source 1..19: mol_type = other DNA
cgaattcgtccacgatgag

SEQ ID NO: 40 - DNA, 18 bp, synthetic construct
  source 1..18: mol_type = other DNA
tcgtcgacaccgagaatg

SEQ ID NO: 41 - DNA, 20 bp, synthetic construct
  source 1..20: mol_type = other DNA
tgtggactgtccgtgcttac

SEQ ID NO: 42 - DNA, 22 bp, synthetic construct
  source 1..22: mol_type = other DNA
agtggttcatctcgaccagatg

SEQ ID NO: 43 - DNA, 22 bp, synthetic construct
  source 1..22: mol_type = other DNA
atgtacacaagtcatcaggatg

SEQ ID NO: 44 - DNA, 20 bp, synthetic construct
  source 1..20: mol_type = other DNA
aggaggtgtttctcgacgtc

SEQ ID NO: 45 - DNA, 21 bp, synthetic construct
  source 1..21: mol_type = other DNA
gtagaaagccagtccgcagaa

SEQ ID NO: 46 - DNA, 20 bp, synthetic construct
  source 1..20: mol_type = other DNA
ggcgggacatagaagtcgta

SEQ ID NO: 47 - DNA, 19 bp, synthetic construct
  source 1..19: mol_type = other DNA
tgtagccgatcacgaggtc

(19) United States
(12) Patent Application Publication
(10) Pub. No.: US 2024/0384226 A1
(43) Pub. Date: Nov. 21, 2024

(54) RHODOPSEUDOMONAS PALUSTRIS TIE-1 STRAINS AND METHODS FOR BIOPLASTIC PRODUCTION

(71) Applicants: Arpita Bose, St. Louis, MO (US); Onina Tahina Ranaivoarisoa, St. Louis, MO (US)

(72) Inventors: Arpita Bose, St. Louis, MO (US); Onina Tahina Ranaivoarisoa, St. Louis, MO (US)

(73) Assignee: Washington University, St. Louis, MO (US)

(21) Appl. No.: 18/666,803

(22) Filed: May 16, 2024

Related U.S. Application Data
(60) Provisional application No. 63/502,474, filed on May 16, 2023.

(51) Int. Cl. C12N 1/20; C12P 7/18; C12R 1/01

(52) U.S. Cl. CPC C12N 1/205; C12P 7/18; C12N 2500/30; C12R 2001/01

(57) ABSTRACT

Transgenic strains of R. palustris TIE-1 and methods of use thereof for bioplastic production are provided. An exemplary transgenic R. palustris TIE-1 microorganism includes an exogenous RuBisCo ΩrubI gene; and an exogenous RuBisCo ΩrubII gene. An exemplary method of producing the bioplastic polyhydroxybutyrate (PHB) includes culturing a transgenic R. palustris TIE-1 microorganism in a culture medium, the transgenic R. palustris TIE-1 microorganism comprising an exogenous RuBisCo ΩrubI gene; and an exogenous RuBisCo ΩrubII gene; such that the exogenous RuBisCo ΩrubI and ΩrubII genes are overexpressed; and recovering the PHB from the culture medium.

### CROSS-REFERENCE TO RELATED APPLICATIONS

This application claims priority from U.S. Provisional Application Ser. No. 63/502,474 filed on 16 May 2023, which is incorporated herein by reference in its entirety.

### STATEMENT REGARDING FEDERALLY SPONSORED RESEARCH OR DEVELOPMENT

This invention was made with government support under MCB2021822 awarded by the National Science Foundation. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020370-US-NP_2024-05-16_Sequence-Listing.xml” created on 16 May 2024; 42,929 bytes). The subject matter of the Sequence Listing is incorporated herein by reference in its entirety.

### FIELD OF THE INVENTION

The present disclosure generally relates to improving the bioplastic, polyhydroxybutyrate (PHB) production in the rhodopseudomonas palustris TIE-1 strain.

### BACKGROUND OF THE INVENTION

Recent improvements in genetic engineering tools have enabled scientists to systematically engineer organisms that produce various value-added chemicals, including biofuels, therapeutic products, food, and bioplastics. At first, most of these engineering efforts were focused on widely used model organisms, such as Escherichia coli, Saccharomyces cerevisiae, and Synechococcus sp. This emphasis resulted in an array of genetic tools that have been effectively developed for valuable biomolecule biosynthesis and for conducting physiological studies. However, the reliance on organic carbon as the primary carbon source poses a limitation for heterotrophic model organisms, contributing to elevated bioproduction costs. Recent studies have highlighted numerous advantages in utilizing non-model organisms for bioproduction. Over the past decade, one such group of microbes that has gained attention is the purple non sulfur bacteria exemplified by Rhodopseudomonas palustris TIE-1 (TIE-1), Rhodospirillum rubrum and Rhodomicrobium. TIE-1, a gram-negative purple non-sulfur photosynthetic bacterium is renowned for its versatile metabolism, rendering it an excellent host for diverse bioproduction and pathway studies. TIE-1 exhibits four primary metabolisms: chemoautotrophy, photoautotrophy, chemoheterotrophy, and photoheterotrophy. These different metabolisms enable TIE-1 to use a wide variety of carbon sources such as carbon dioxide (CO₂) and many organic acids. TIE-1 also can use ammonium salt such as ammonium chloride (NH₄Cl) or fix nitrogen from dinitrogen gas (N₂) as a nitrogen source. Moreover, it can use multiple electron sources including hydrogen (H₂) or ferrous iron Fe(II). One of the most appealing features of TIE-1 is its ability to uptake electrons directly from a poised electrode, which enables one to use it in microbial electrosynthesis (MES). MES is a system in which microorganisms are used to produce valuable compounds using their ability to exchange electrons from the bioelectrical reactor. Using electrons from a poised electrode or a solar panel-powered MES system, TIE-1 produced biodegradable plastic and biofuel using CO₂ as a carbon source, N₂ as a nitrogen source, and light as an energy source. These represented the first steps toward a sustainable and carbon-neutral process for bioproduction using TIE-1 in MES. Besides its ability to utilize various substrates, TIE-1's metabolic diversity also makes it an extraordinary model organism for pathway investigation. For example, the use of RuBisCo mutants allows one to study the link between the Calvin-Benson-Bassham (CBB) cycle in carbon fixation and extracellular electron transport. Similarly, a TIE-1 pioABC mutant was used to investigate the electron uptake mechanism during photoferrotrophic and electrotrophic growth conditions. During these studies, mutants were first generated and grown under heterotrophic growth conditions in which neither the RuBisCo nor the electron uptaking machineries were involved. These strains were then switched to autotrophic and electron uptaking growth conditions to further understand their role in metabolism. Not only do these studies provide insight into TIE-1's metabolism, but they also open doors for a deeper understanding of other closely related purple non-sulfur bacteria, such as CGA009 that has been studied extensively for biohydrogen production. The available genetic tools for TIE-1 are limited compared to widely used model organisms, with most tools being based on homologous recombination.

### SUMMARY

The present disclosure describes improvement to the production of bioplastic polyhydroxybutyrate (PHB) from Rhodopseudomonas palustris TIE-1 (Rpal TIE-1) using gene engineering including: mutants lacking the glycogen gene (gly): (e.g., ΔRpal_(Rpal_0386)); double mutants lacking the nitrogenase regulator nifA genes (comprising nifA1 and nifA2 (e.g., Rpal_1624 and Rpal_5113)); mutants lacking the regulator phaR (Rpal_0531) of the PHB gene; mutants lacking the PHB depolymerase phaZ (Rpal_0568); and/or engineered TIE-1 strains that overexpress RuBisCo forms I and Il when integrated in the genome.

The transgenic TIE-1 strains disclosed herein sequester CO2 and turn it into bioplastic (e.g., PHB), thereby increasing bioplastic production as compared to wild-type TIE-1 and at the same time reducing CO2 emission during the reaction/production process.

Some features of the present disclosure teach a transgenic R. palustris microorganism comprising at least one exogenous RuBisCo gene. In some embodiments, the exogenous RuBisCo gene is selected from ΩrubI, ΩrubII, and combinations thereof. In some embodiments, the exogenous RuBisCo gene is integrated into the genome of the microorganism and driven by a constitutively strong promoter PaphII using a φC31 integrase system. In some embodiments, the microorganism does not comprise a functional copy of at least one endogenous gene selected from the group consisting of: glgA, nifA1, nifA2, phaR, phaZ, and combinations thereof. In some embodiments, the transgenic R. palustris microorganism is capable of increased bioplastic production compared to a wild-type R. palustris microorganism. In some embodiments, the transgenic R. palustris microorganism is derived from R. palustris TIE-1.

Other features of the present disclosure teach a method of producing polyhydroxybutyrate (PHB), the method comprising: culturing a transgenic microorganism according to any one of the preceding claims in a culture medium; and recovering the PHB from the culture medium.

In an aspect of the present disclosure, a transgenic R. palustris TIE-1 microorganism is provided. The transgenic R. palustris TIE-1 microorganism comprising: an exogenous RuBisCo ΩrubI gene; and an exogenous RuBisCo ΩrubII gene.

In some embodiments, the transgenic R. palustris TIE-1 microorganism further comprises an endogenous gene phaR deletion, such that the transgenic R. palustris TIE-1 microorganism lacks a functional copy of a phaR gene. In some embodiments, the transgenic R. palustris TIE-1 microorganism further comprises an endogenous gene phaZ deletion, such that the transgenic R. palustris TIE-1 microorganism lacks a functional copy of a phaZ gene. In some embodiments, the transgenic R. palustris TIE-1 microorganism further comprises an endogenous gene phaR deletion, such that the transgenic R. palustris TIE-1 microorganism lacks a functional copy of a phaR gene; and an endogenous gene phaZ deletion, such that the transgenic R. palustris TIE-1 microorganism lacks a functional copy of: a phaR gene and a phaZ gene. In some embodiments, the transgenic R. palustris TIE-1 microorganism further comprises an endogenous gene gly deletion, such that the transgenic R. palustris TIE-1 microorganism lacks a functional copy of a gly gene. In some embodiments, the transgenic R. palustris TIE-1 microorganism further comprises an endogenous gene nifA deletion, such that the transgenic R. palustris TIE-1 microorganism lacks a functional copy of a nifA gene. In some embodiments, the endogenous gene nifA deletion comprises deletion of a nifA1 gene and a nifA2 gene. In some embodiments, the exogenous RuBisCo ΩrubI and ΩrubII genes are integrated into a genome of the transgenic R. palustris TIE-1 microorganism using a φC31 integrase system. In some embodiments, the transgenic R. palustris microorganism is capable of increased bioplastic production compared to a wild-type R. palustris microorganism.

In another aspect of the present disclosure, a method of producing polyhydroxybutyrate (PHB) is provided. The method comprising: culturing a transgenic R. palustris TIE-1 microorganism in a culture medium, wherein the transgenic R. palustris TIE-1 microorganism comprises: an exogenous RuBisCo ΩrubI gene; and an exogenous RuBisCo ΩrubII gene; such that the exogenous RuBisCo ΩrubI and ΩrubII genes are overexpressed; and recovering the PHB from the culture medium.

In some embodiments, the exogenous RuBisCo ΩrubI and ΩrubII genes are integrated into a genome of the transgenic R. palustris TIE-1 microorganism using a φC31 integrase system. In some embodiments, the culture medium comprises photoheterotrophic conditions with butyrate and nitrogen; photoheterotrophic conditions with butyrate and ammonium chloride; photoautotrophic conditions with hydrogen and ammonium chloride; or photoelectrotrophic conditions with nitrogen.

In a further aspect of the present disclosure, a method of producing polyhydroxybutyrate (PHB) is provided. The method comprising: culturing a transgenic R. palustris TIE-1 microorganism in a culture medium, wherein the transgenic R. palustris TIE-1 microorganism comprises: an exogenous RuBisCo ΩrubI gene and an exogenous RuBisCo ΩrubII gene, such that the exogenous RuBisCo ΩrubI and ΩrubII genes are overexpressed; an endogenous gene phaR deletion, such that the transgenic R. palustris TIE-1 microorganism lacks a functional copy of a phaR gene; and an endogenous gene phaZ deletion, such that the transgenic R. palustris TIE-1 microorganism lacks a functional copy of a phaZ gene; and recovering the PHB from the culture medium.

In some embodiments, the exogenous RuBisCo ΩrubI and ΩrubII genes are integrated into a genome of the transgenic R. palustris TIE-1 microorganism using a φC31 integrase system. In some embodiments, the culture medium comprises photoheterotrophic conditions with butyrate and nitrogen; photoheterotrophic conditions with butyrate and ammonium chloride; photoautotrophic conditions with hydrogen and ammonium chloride; or photoelectrotrophic conditions with nitrogen.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic of the φC31 integrase mechanism. Plac: Plac promoter, PaphII: PaphII promoter, mcherry: red fluorescent protein, genR: gentamicin resistance, p15A: the origin of replication, pBBR1: broad host origin of replication attP: attP site for φC31 integrase, attB: attB site for φC31 integrase, φC31: φC31 integrase.

FIG. 1B is a schematic of the plasmid based integration system. Plac: Plac promoter, PaphII: PaphII promoter, mcherry: red fluorescent protein, genR: gentamicin resistance, p15A: the origin of replication, pBBR1: broad host origin of replication attP: attP site for φC31 integrase, attB: attB site for φC31 integrase, φC31: φC31 integrase.

FIG. 1C is a schematic of the genome-based integration system. Plac: Plac promoter, PaphII: PaphII promoter, mcherry: red fluorescent protein, genR: gentamicin resistance, p15A: the origin of replication, pBBR1: broad host origin of replication attP: attP site for φC31 integrase, attB: attB site for φC31 integrase, φC31: φC31 integrase.

FIG. 2A is a graph of the transformation efficiency normalized to the plasmid amount. TE: transformation efficiency, PC: plasmid-based system with the constitutive promoter, PI: plasmid-based system with the inducible promoter, GI: genome-based system with the inducible promoter, CFU: colony forming units, OD: optical density

FIG. 2B is a graph of editing efficiency. TE: transformation efficiency, PC: plasmid-based system with the constitutive promoter, PI: plasmid-based system with the inducible promoter, GI: genome-based system with the inducible promoter, CFU: colony forming units, OD: optical density

FIG. 3A is a graph of growth of all mutant strains under non-nitrogen fixing conditions.

FIG. 3B is a graph of growth of all mutant strains under nitrogen-fixing conditions (N2).

FIG. 3C is a graph of growth of the RuBisCo engineered strains under non-nitrogen fixing conditions.

FIG. 3D is a graph of growth of all the RuBisCo engineered stains under nitrogen-fixing conditions.

FIG. 3E is a graph of PHA production from mutants and TIE strains grown with butyrate under non-nitrogen and nitrogen fixing conditions.

FIG. 3F is a graph of PHA production from the RuBisCo engineered and wild type TIE-1 strains grown with butyrate under non-nitrogen and nitrogen fixing conditions. The statistical differences in PHA production were calculated using two-tailed Student's test against wild type TIE-1. *P≤0.05, **P<0.01, ***P<0.001.

FIG. 4A is a graph of growth of all the mutant strains under non-nitrogen fixing conditions.

FIG. 4B is a graph of growth of all the mutant strains under nitrogen-fixing conditions.

FIG. 4C is a graph of growth of the engineered RuBisCo strains under non-nitrogen fixing conditions.

FIG. 4D is a graph of growth of all the engineered RuBisCo stains under nitrogen-fixing conditions.

FIG. 4E is a graph of PHA production from mutants and TIE-1 wild type strains grown under non-nitrogen or nitrogen fixing conditions.

FIG. 4F is a graph of PHA production from RuBisCo engineered and wild type strains grown under hydrogen under non-nitrogen or nitrogen fixing conditions. The statistical differences in PHA production were calculated using two-tailed Student's test against the wild type TIE-1. *P≤0.05, **P<0.01 ***P<0.001. NS=not significant, ND=Not detected, and NG=No growth. Error bars represent standard errors from biological triplicates.

FIG. 5A is a graph of Fe(II) concentration variation from the mutants grown under non-nitrogen fixing conditions.

FIG. 5B is a graph of Fe(II) concentration variation from the mutans during nitrogen-fixing conditions with N₂.

FIG. 5C is a graph of growth of the engineered RuBisCo strains with under non-nitrogen fixing conditions.

FIG. 5D is a graph of growth of all the engineered RuBisCo stains under nitrogen-fixing growth conditions.

FIG. 5E is a graph of PHA productivity from mutants grown under non-nitrogen fixing conditions nitrogen fixing conditions photoferrotrophically.

FIG. 5F is a graph of PHA production from engineered RuBisCo strains grown under non-nitrogen or nitrogen-fixing conditions. The statistical differences in PHA production were calculated using two-tailed Student's test against the wild type TIE-1. *P≤0.05, **P<0.01, ***P<0.001. NS=not significant, ND=Not detected, and NG=No growth. Error bars represent standard errors from biological triplicates.

FIG. 6A is a graph of current density from the wild type TIE-1 (WT) strain, the various mutants ΔphaR, ΔphaZ, Δgly, ΔnifA DM (double mutant) and the engineered strains Ωrub(I) and Ωrub(I&II) under non-nitrogen fixing growth conditions.

FIG. 6B is a graph of current uptake from the wild type TIE-1 (WT) strain, the various mutants ΔphaR, ΔphaZ, Δgly, ΔnifA DM (double mutant) and the engineered strains Ωrub(I) and Ωrub(I&II) under non-nitrogen fixing growth conditions.

FIG. 6C is a graph of current density from the wild type TIE-1 (WT) strain, the various mutants ΔphaR, ΔphaZ, Δgly, ΔnifA DM (double mutant) and the engineered strains Ωrub(I) and Ωrub(I&II) under nitrogen fixation conditions (N2).

FIG. 6D is a graph of current uptake from the wild type TIE-1 (WT) strain, the various mutants ΔphaR, ΔphaZ, Δgly, ΔnifA DM (double mutant) and the engineered strains Ωrub(I) and Ωrub(I&II) under nitrogen fixation conditions (N₂).

FIG. 6E is a graph of PHA production from mutants grown under non-nitrogen and nitrogen fixing conditions photoelectrotrophically.

FIG. 6F is a graph of PHA productivity from engineered RuBisCo strains grown under photoelectrotrophic non-nitrogen and nitrogen fixing growth conditions. The statistical differences in PHA production were calculated using two-tailed Student's test against the wild type TIE-1. *P≤0.05, **P<0.01, ***P<0.001. NS=not significant, ND=Not detected, and NG=No growth. Error bars represent standard errors from biological triplicates.

FIG. 7 is a table of the strains used in the disclosure described herein.

FIG. 8 is a table of the plasmids used in the disclosure described herein.

FIG. 9 is a table of the growth parameters values of the knockouts and engineered TIE-1 strains grown under different growth conditions.

FIG. 10 is a table of PHA production from different TIE-1 strains under photoautotrophic and heterotrophic growth conditions.

FIG. 11 is a table of current uptake obtained from the different strains during photoelectroautotrophic growth under non-nitrogen or nitrogen fixing conditions.

FIG. 12A is a schematic of generating a knockout mutant. UP: upstream homologous arm, DN: down-stream homologous arm, Genr: gentamicin resistance, SacB: sucrose counter-selection marker.

FIG. 12B is a schematic of generating a knock-in mutant. UP: upstream homologous arm, DN: down-stream homologous arm, Genr: gentamicin resistance, SacB: sucrose counter-selection marker.

FIG. 13A is a gel image showing the expected sizes from the primers sets used for checking ΔphaR. Mut=mutant, WT=wild type.

FIG. 13B is a gel image showing the expected sizes bands for checking ΔphaZ. Mut=mutant, WT=wild type.

FIG. 14 is a schematic of the creation of the two newly constructed pJQ200KS plasmids with gentamycin or kanamycin cassette Gentamycin (GmR) cassette of the pJQ200KS plasmid that was replaced with either a kanamycin gene resistance cassette (KanR) or a chloramphenicol gene resistance cassette (CmR). MCS—Multiple Cloning Site. Pc-Pc—Pc promoter.

FIG. 15 is an image showing the Ωrub(I) and Ωrub(I&II) engineered strains using PCR amplification spanning the promoter region PaphII to the integrated either RuBisCo form I (rubI) or form II (rubII). Expected band: Lane 1˜800 bp, Lane 2=0 bp, Lane 3=empty, Lane 4˜800 bp, Lane 5˜1500 bp, Lan 6=0 bp. The primers set used for the PCR test are shown at the bottom of the figure and listed in FIG. 19.

FIG. 16 is a table of the list of antibiotics and sucrose used in the described study and their respective concentration.

FIG. 17 is a table of the list of primers used to design plasmids used in the studies described herein.

FIG. 18 is a table of the list of primers used to design and check the mutants constructed in this study.

FIG. 19 is a table of the primers used to check the insertion of RuBisCo I and II in TIE-1 used in this study.

FIG. 20 is a graph of PHB production from the RuBisCo engineered strains and wild type TIE-1 strains grown under hydrogen with NH₄Cl or N₂.

FIG. 21 is a graph of PHB the multiples mutants and TIE-1 wild type strains grown under hydrogen with NH₄Cl or N₂.

FIG. 22 is a graph of growth of the different strains from hydrogen with NH₄Cl.

FIG. 23 is a graph of growth of the different strains from hydrogen with N₂.

FIG. 24 is a graph of growth of all strains with butyrate.

FIG. 25 is a graph of PHB production obtained from the mutants grown with butyrate.

FIG. 26 is a graph of PHB production obtained from the engineered strains grown with butyrate.

FIG. 27 is a graph of cell density as OD660 of TIE-1 & RuBisCo engineered strains grown under photoelectroautotrophy with NH₄Cl.

FIG. 28 is a graph of PHB produced by TIE-1 & RuBisCo engineered strains grown under photoelectroautotrophy with NH₄Cl.

FIG. 29 is a graph of current uptake vs Time profile of TIE-1 & RuBisCo engineered strains for PHB synthesis at poised potential of +100 mV vs. SHE.

FIG. 30 is a graph of current uptake vs Time profile of TIE-1 & RuBisCo engineered strains for PHB synthesis at poised potential of +100 mV vs. SHE.

### DETAILED DESCRIPTION OF THE INVENTION

The present disclosure is based, at least in part, on a design to improve the bioplastic polyhydroxybutyrate (PHB) from Rhodopseudomonas palustris TIE-1 using gene engineering. This includes, but is not limited to, mutant lacking the glycogen gene: ΔRpal_(Rpal_0386), double mutant lacking the nitrogenase regulator nifA1 and nifA2: Rpal_1624 and Rpal_5113, mutant lacking the regulator phaR (Rpal_0531) of the PHB gene, mutant lacking the PHB depolymerase phaZ (Rpal_0568), and engineered TIE-1 strains that have to overexpress RuBisCo from I and II integrated in the genome.

In some aspects, these discoveries aim to lower bioplastic production and at the same time reduce CO₂ emission. In another aspect, Rpal TIE-1 can sequester CO2 and turn it into bioplastic. In another aspect, being biodegradable, transitioning to bioplastic can also tremendously reduce the pollution caused by conventional plastic. As shown herein, the φC31 integration system was used to integrate additional copies of the RuBisCo gene form I and II driven by PaphII constitutive promoter into TIE-1 genome. Additionally, to increase PHA accumulation in TIE-1, mutants were created that lack key genes in PHA pathway particularly focusing on the phaZ and the phaR genes. The behavior of the mutants lacking the phaR helped to elucidate whether phaR is an activator or repressor of PHA pathway in TIE-1. In addition to exploring genes within PHA pathway, the impact of deleting genes in pathways that might potentially compete with PHA synthesis pathway were also examined. These include the mutants that have been previously studied in biobutanol production in TIE-1: Δgly mutant lacking glycogen synthase and the mutant lacking the two NifA regulators nifA; Rpal_1624 & Rpal_5113 of the nitrogen fixation pathway of TIE-1. In some aspects, like R. eutropha, overexpressing RuBisCo genes (form I and II) in TIE-1 can increase intracellular carbon abundance, and hence increase PHA accumulation. PHA production was tested in all six strains and wild type under a variety of growth conditions, including non-nitrogen fixing conditions with NH₄Cl (referred to as non-nitrogen fixing conditions throughout) and nitrogen-fixing conditions with N₂ gas (referred to as nitrogen-fixing conditions throughout).

The results of the current disclosure show that deletion of phaR gene increased PHA production (cell % v/v) when TIE-1 was grown photoheterotrophically with butyrate under non-nitrogen fixing conditions. An increase in PHA production (cell % v/v) from the Δgly and ΔnifA under photoautotrophic growth conditions with H₂ and under non-nitrogen fixing conditions was also observed. PHA production increased in TIE-1 strains overexpressing RuBisCo genes form I and form I&II under photoheterotrophy with butyrate irrespective of the nitrogen source used and photoautotrophy with H₂ under non-nitrogen fixing conditions as well as photoelectrotrophically under nitrogen fixing conditions. It is shown that both gene deletion and overexpression can enhance PHA production by TIE-1. This disclosure advances TIE-1 as a model organism for potential commercialization for PHA production and provides valuable insights for future genetic engineering endeavors aimed at enhancing bioplastic production in other purple non sulfur bacteria.

In some aspects, synthetic biology can be used to improve PHB productivity in TIE-1. In another aspect, the overexpression of the two RuBisCo form I only or both form I and II did not result in the same effect. More specifically, the effect of the overexpression of the RuBisCo gene form I and II on PHB productivity appears to be cumulative at least from the result obtained under photoheterotrophic with butyrate. That is to say overexpression of form I only resulted in less increase whereas the overexpression of both forms I and II gave higher PHB productivity.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase Il type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA: DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA: DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253). Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, TIE-1 signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of TIE-1 by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome.

For example, the methods as described herein can comprise a method for altering a target polynucleotide sequence in a cell comprising contacting the polynucleotide sequence with a clustered regularly interspaced short palindromic repeats-associated (Cas) protein.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Overexpression of Rubisco Form I and II in Rhodopseudomonas Palustris TIE-1 Augments 2 Polyhydroxyalkanoate Production Heterotrophically and Autotrophically

With the increasing demand for sustainably produced renewable resources, it is important to look towards microorganisms capable of producing bioproducts such as bioplastics. Though many systems for bioproduction are well documented and evaluated in model organisms, it is essential to look beyond non-model organisms to expand the field and take advantage of metabolically versatile strains. Example 1 centers on Rhodopseudomonas palustris TIE-1, a purple, non-sulfur autotrophic, and anaerobic bacterium capable of producing bioplastics comparable to their petroleum-derived counterparts. To increase bioplastic production, genes potentially involved in polyhydroxyalkanoate (PHA) biosynthesis, including the regulatory gene phaR and phaZ encoding PHA granule-degrading enzyme were deleted in TIE-1. Moreover, genes associated with pathways that might compete with PHA production, specifically those linked to glycogen and nitrogen fixation, were eliminated using a markerless deletion method. Additionally, RuBisCo form I and II genes were integrated into TIE-1 genome by a phage integration system, developed in this study. This system enables precise and rapid gene integration in TIE-1. The results show that deletion of phaR increases PHA production when TIE-1 is grown photoheterotrophically with butyrate and ammonium chloride (NH₄Cl). Mutants unable to make glycogen or fix dinitrogen gas (N₂) show increased PHA production under photoautotrophic growth conditions with hydrogen and NH₄Cl. The overexpression of RuBisCo form I and form I&II increased PHA production under photoheterotrophy with butyrate by three to five times, and two times under photoautotrophy with hydrogen and NH₄Cl as well as photoelectrotrophic growth with N₂. In summary, inserting RuBisCo genes into the TIE-1 genome is a more effective strategy than deleting competitive pathways to increase PHA production in TIE-1. Using RuBisCo genes, it is also demonstrated that the phage integration system developed for TIE-1 creates numerous opportunities for synthetic biology in TIE-1.

The planet has been burdened by pollution resulting from the extensive use of petroleum-derived plastics for the last few decades. Since the discovery of biodegradable plastic alternatives, concerted efforts have been made to enhance their bioproduction. The versatile microorganism Rhodopseudomonas palustris TIE-1 (TIE-1) stands out as a promising candidate for bioplastics synthesis, owing to its ability to use multiple electron sources, fix the greenhouse gas CO₂, and use light as an energy source. Two categories of strains were meticulously designed from the TIE-1 wild type to augment polyhydroxyalkanoate (PHA) production. The first group includes mutants carrying a deletion of the phaR or phaZ genes in PHA pathway, and those lacking potential competitive carbon and energy sources to PHA pathway (namely, glycogen biosynthesis and nitrogen-fixation). The second group comprises TIE-1 strains that overexpress RuBisCo form I or form I&II genes inserted via a phage integration system. By studying numerous metabolic mutants and overexpression strains, it is concluded that genetic modifications in the environmental microbe TIE-1 can improve PHA production. When combined with other approaches (such as reactor design, use of microbial consortia, and different feedstocks), genetic and metabolic manipulation of purple non sulfur bacteria like TIE-1 are essential for replacing petroleum-derived plastics with biodegradable plastics like PHA.

### Results

Generating TIE-1 Mutants and Suicide Plasmid with the Different Antibiotic Marker

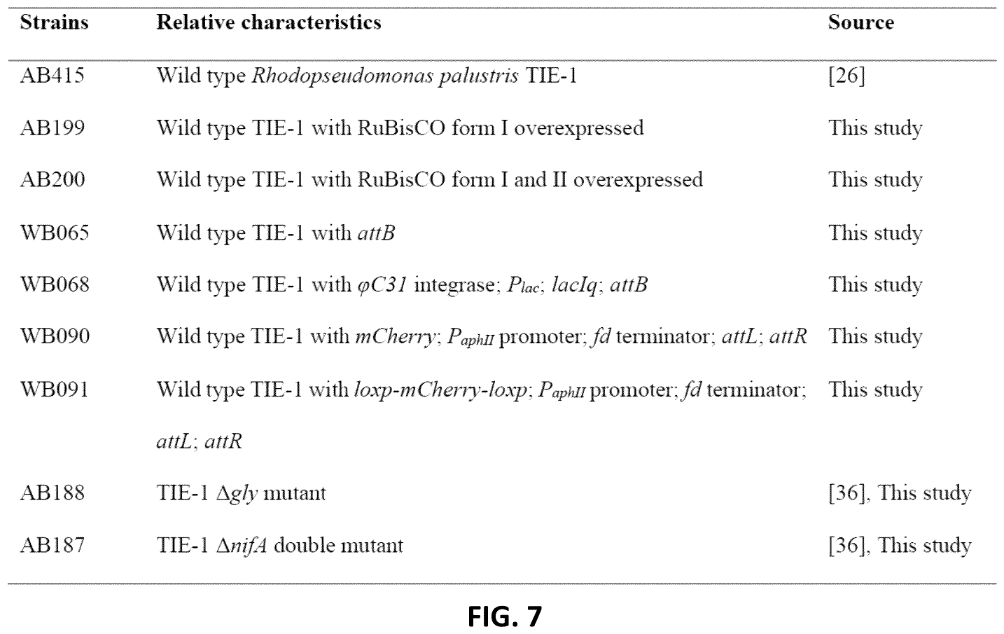

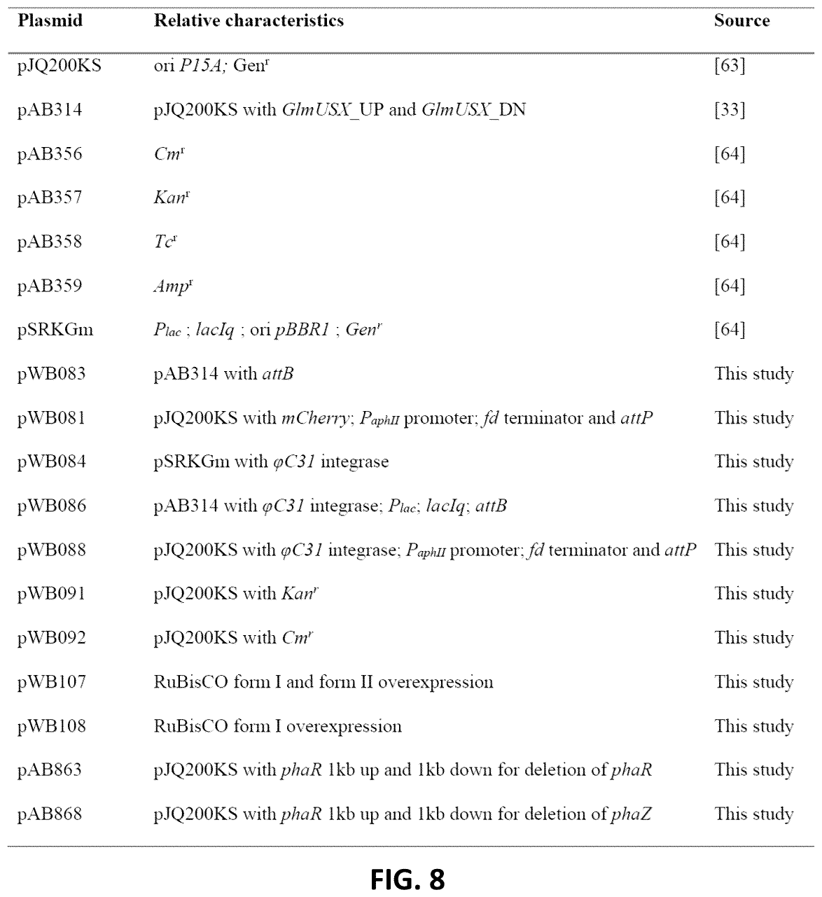

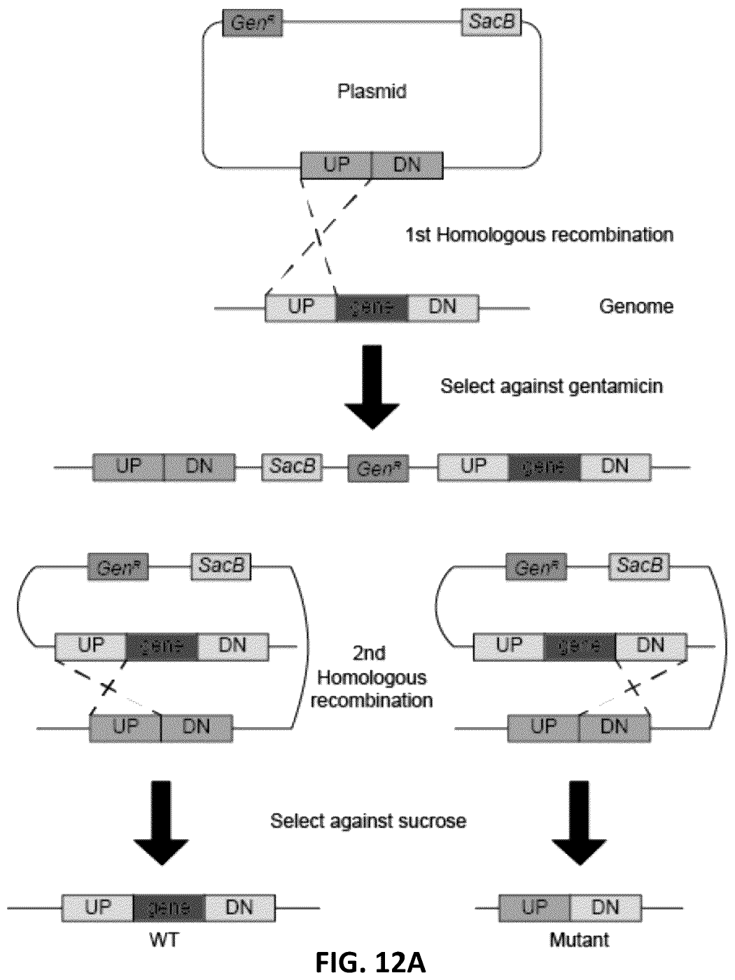

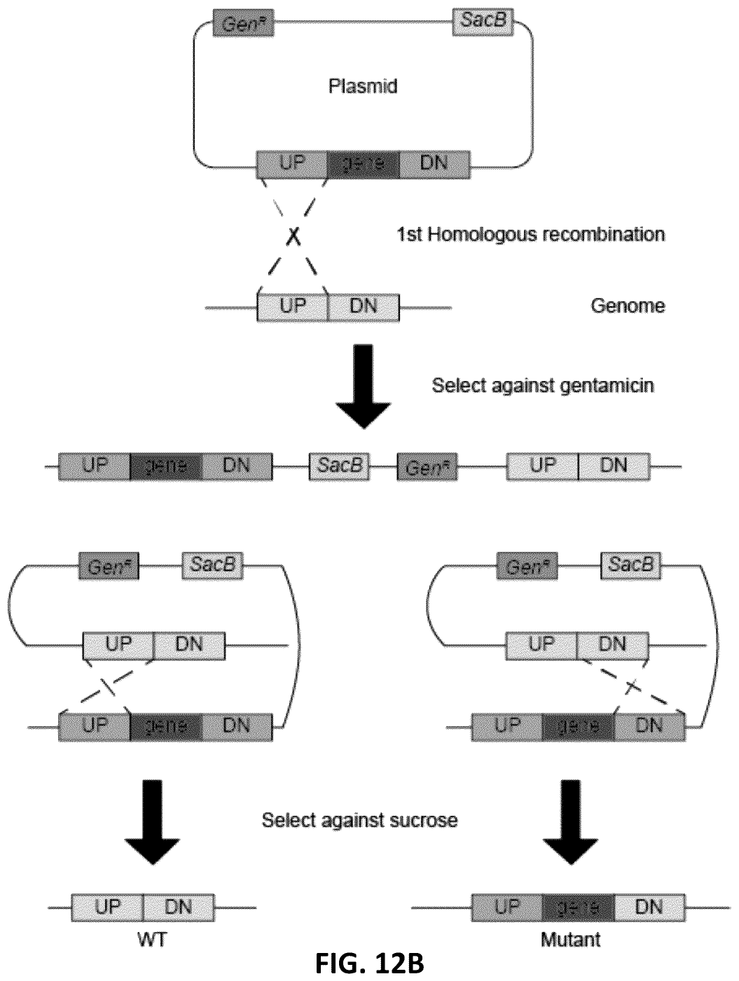

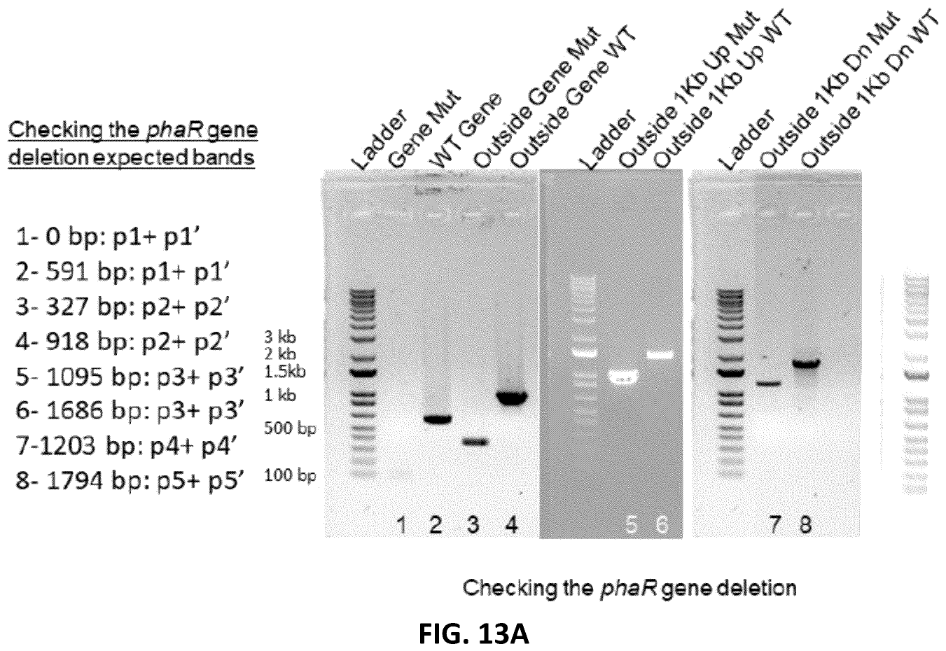

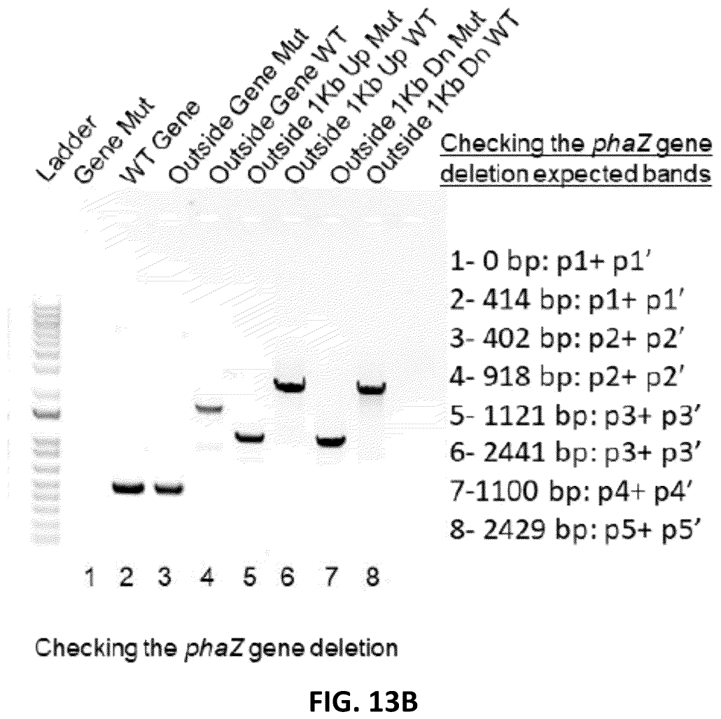

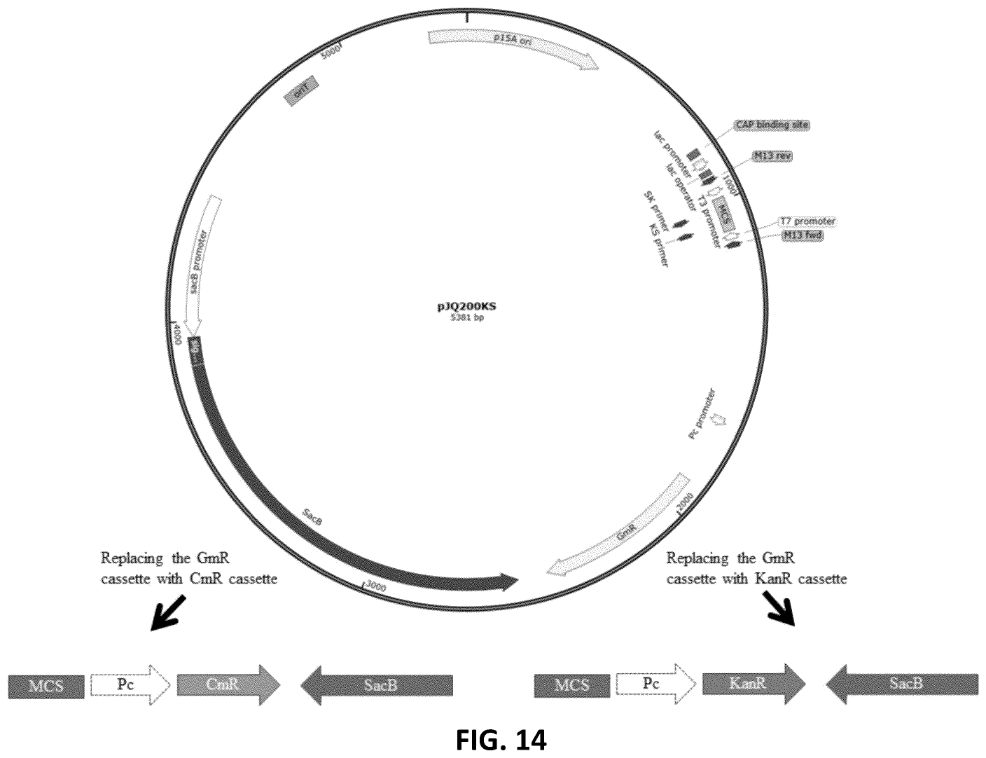

To improve polyhydroxyalkanoate (PHA) production, a mutant lacking the regulator of PHA pathway (ΔphaR) was constructed, and a mutant lacking PHA depolymerase (ΔphaZ) (FIGS. 12 and 13). The mutant lacking the glycogen gene (Δgly), as well as the double mutant lacking the nitrogen-fixing regulator nifA genes (ΔnifA1 ΔnifA2; referred as ΔnifA throughout) were used as is. These strains are listed FIG. 7 and the plasmids used to construct them are listed in FIG. 8. All the mutants were constructed using a suicide plasmid (pJQ200KS) carrying a gentamicin resistance cassette. Using gentamycin has two disadvantages, 1) TIE-1 has a remarkably high resistance to gentamicin with a minimum inhibitory concentration (MIC) of 200 μg/mL, 2) gentamicin is expensive. For enhanced competitiveness in large-scale PHA production, opting for other antibiotics with lower MIC values than gentamicin could offer a more cost-effective alternative. Accordingly, suicide plasmids containing two widely used antibiotic resistance markers: chloramphenicol (MIC of 100 μg/mL) or kanamycin (MIC of 50 μg/mL), using pJQ200KS as the backbone as listed in FIG. 8 and shown in FIG. 14.

### Engineering TIE-1 Using a Phage Integration System to Increase PHA Production in TIE-1

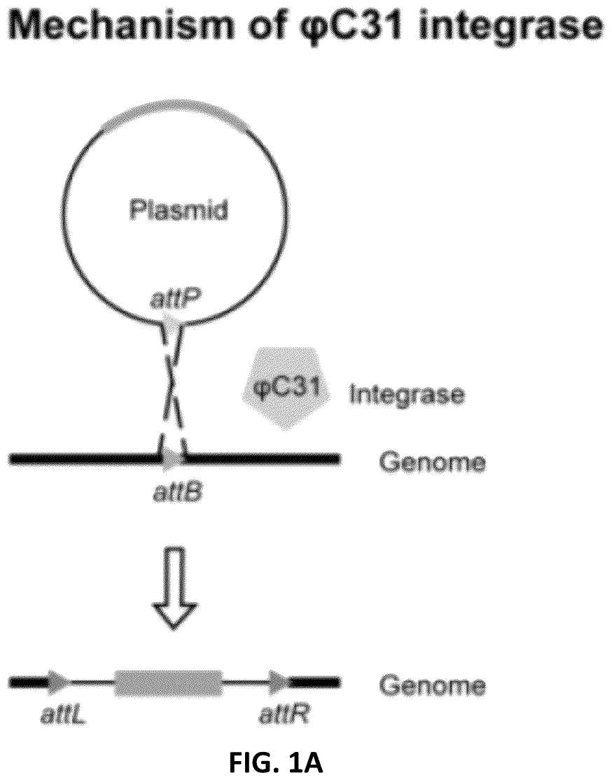

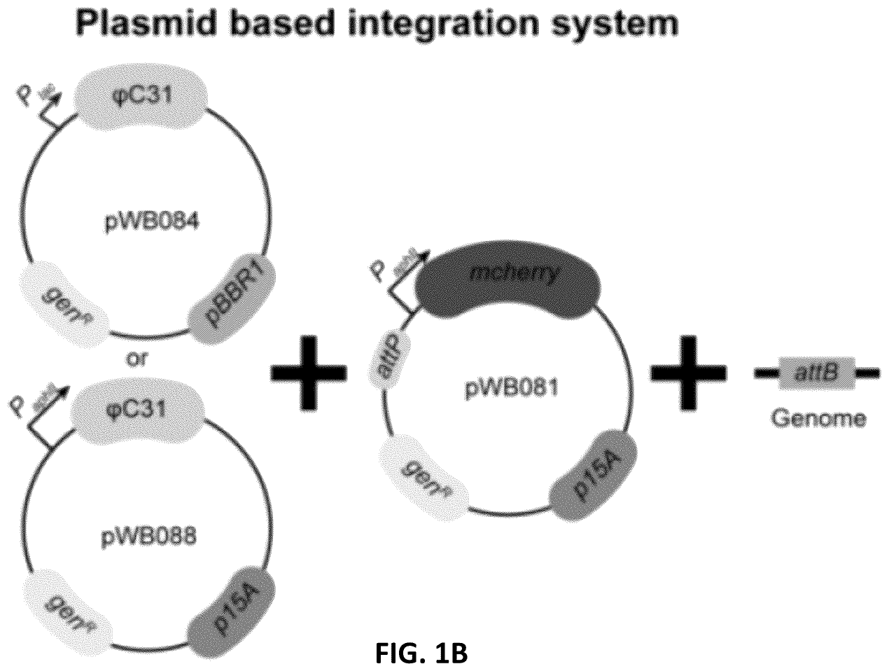

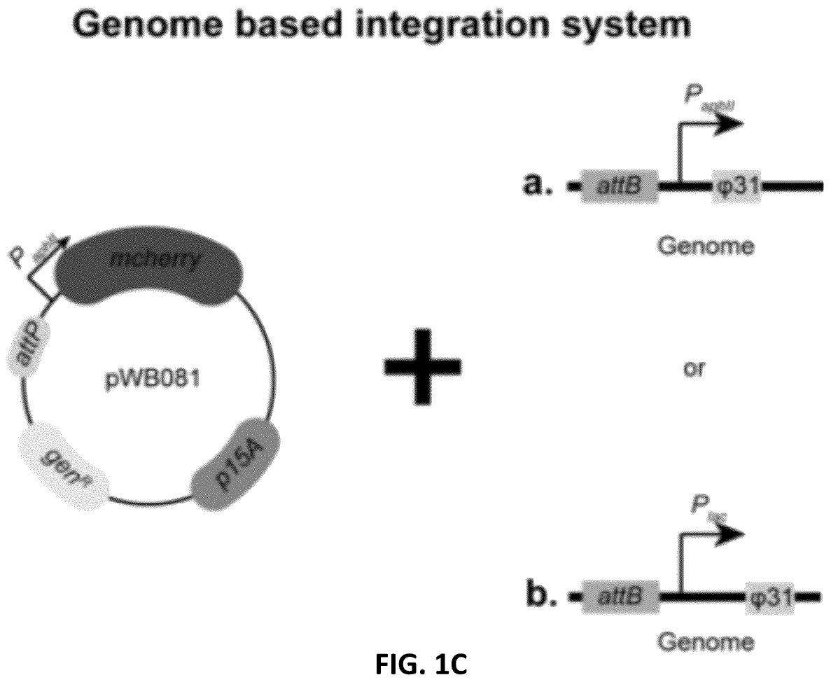

As previously discussed, homologous recombination presents both a time-intensive process and remains so far, the only successful genetic tool for TIE-1. To address these challenges, a phage integration system for gene incorporation into its genome was designed. FIG. 1 delineates the essential components of the φC31recombinase system: the attB site, attP site, and the φC31 integrase.

To compensate for the lack of an attB site in TIE-1, it was inserted into the genome as described in the Materials and Methods section. The attP site was introduced into a suicide plasmid with a constitutively expressed mCherry gene under the PaphII promoter (pWB081). For the expression of φC31 integrase, the optimal way would be to use a temperature-sensitive plasmid. After the targeted genome editing, the removal of the φC31 integrase could be conveniently executed using this approach. Unfortunately, there is no known temperature-sensitive plasmid that replicates in TIE-1. Hence, it was decided to build two different systems: (1) a plasmid-based system, where the integrase is introduced into TIE-1 by a plasmid (FIG. 1B), and (2) a genome-based system, where the integrase is integrated into the TIE-1 genome (FIG. 1C). The advantage of the plasmid-based system is its mobility, while the genome-based system is more stable and does not rely on antibiotics. Both an inducible promoter (Plac) and a strong constitutive promoter (PaphII) were tested. To summarize, as shown in FIG. 1, there are four different designs for the expression of the φC31 integrase: a) PaphII-driven φC31 integrase on a suicide plasmid (pWB088); b) Plac-driven φC31 integrase on a self-replicating plasmid (pWB084); c) PaphII-driven φC31 integrase on TIE-1 genome (FIG. 1C, upper right); and d) Plac-driven φC31 integrase on TIE-1 genome (FIG. 1C, lower right).

After three separate trials, a genome-based system using the constitutive promoter (FIG. 1C, upper right) was not obtained, which indicated that the strong constitutive expression of φC31 integrase is probably lethal to TIE-1. Thus, the results of the other three systems are presented. The successful integration of mCherry was indicated by visualizing red fluorescence at an emission of 610 nm.

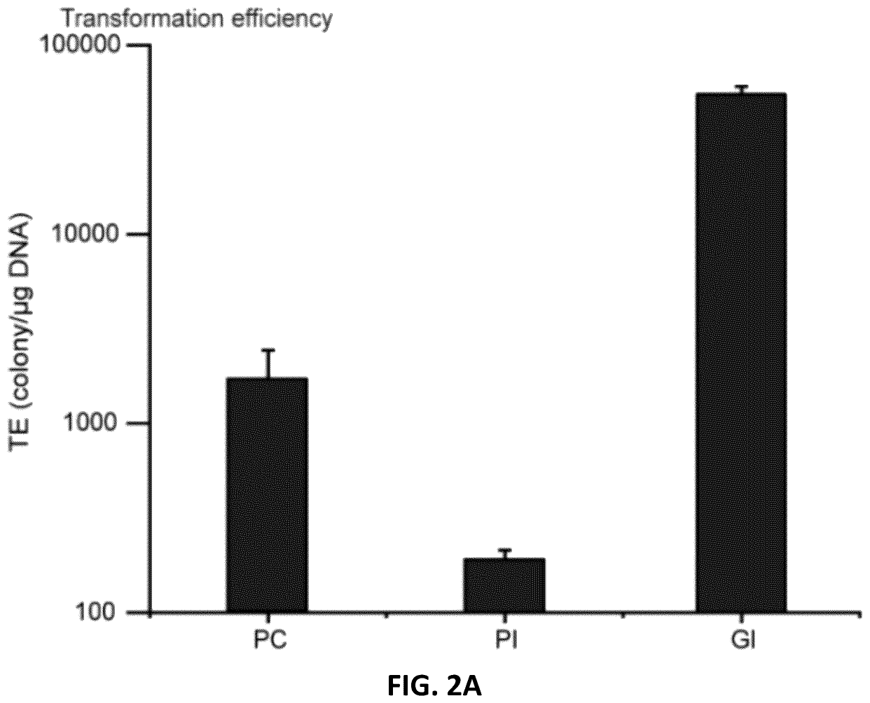

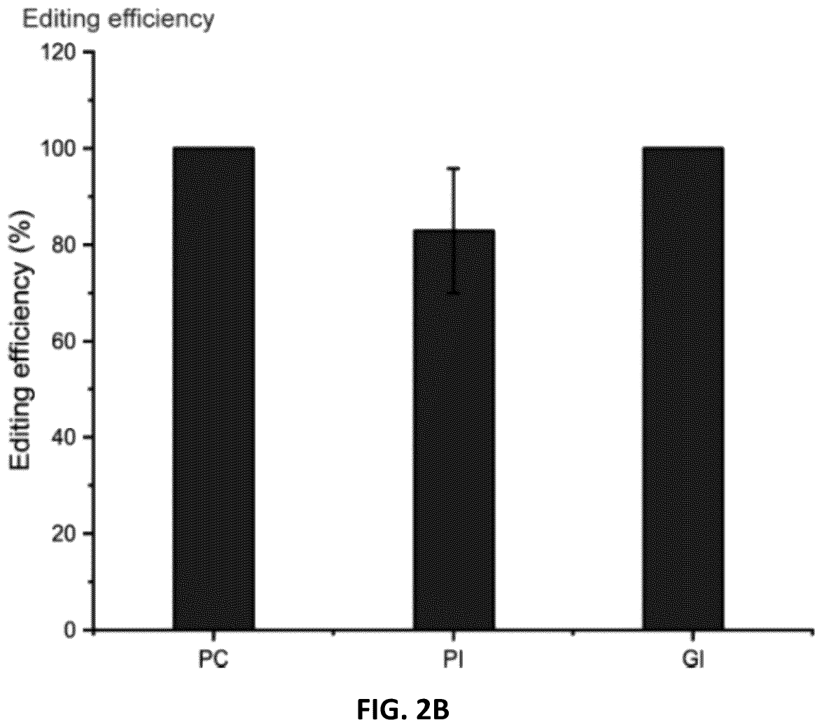

Upon confirming integration, the efficiency of the various systems were assessed by measuring transformation efficiency (detailed calculation described in the Materials and Methods section) and the integration efficiency, which is defined as the percentage of colonies that have red fluorescence signals among all obtained colonies. As shown in FIGS. 2A and 2B, the transformation efficiency normalized to the plasmid concentration is higher for the genome-based system (FIG. 1C). This higher efficiency could be due to the sufficiency of only one plasmid for the system to be functional. Between the two plasmid-based systems, the constitutively expressed φC31 reached higher efficiency (FIG. 1B, pWB088). This higher efficiency could be due to the independence from the use of the inducer, IPTG. For the integration frequency, the genome-based system (FIG. 1C) and the plasmid-based system with the constitutive promoter (FIG. 1B, pWB088) resulted in editing efficiencies of 100% (FIG. 2B). However, the plasmid-based system with the inducible promoter only reached an editing efficiency of about 80% (FIG. 2B). In summary, the genome-based system and constitutive promoter result in higher transformation efficiency and genome editing efficiency.

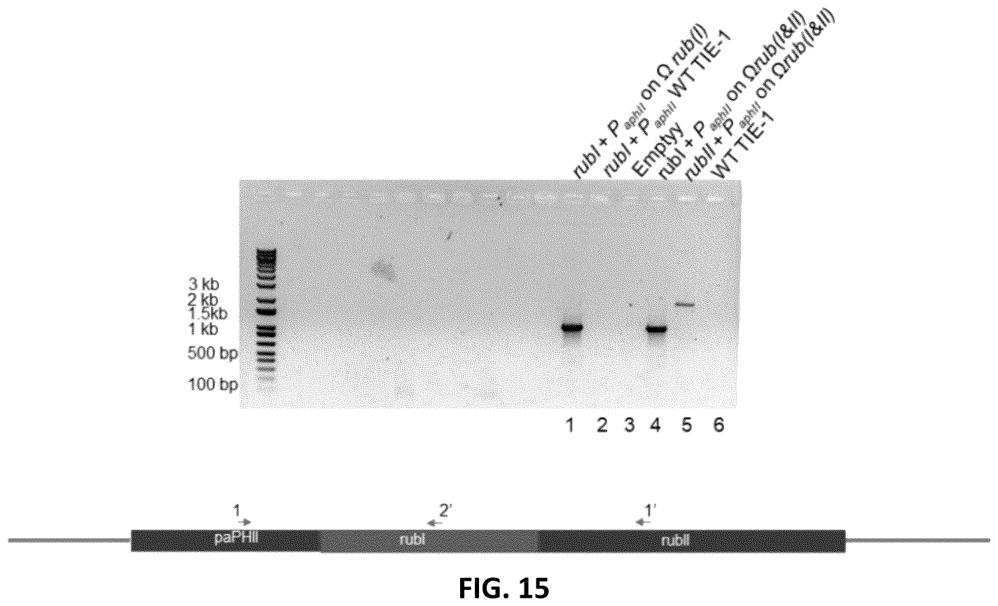

It has been shown in Ralstonia eutropha (now C. necator) that overexpression of RuBisCo resulted in higher PHA production. Thus, to improve PHA production, a PaphII-driven RuBisCo form I alone or RuBisCo form I and form II together into the TIE-1 genome was integrated to obtain two new TIE-1 strains: Ωrub(I) and Ωrub(I&II). The successful integration of these genes and PaphII was checked by PCR amplifying the PaphII region and the RuBisCo gene form I and II as indicated in FIG. 15.

Deletion of phaR, phaZ, or Overexpression of the Rubisco Form I Impaired the Growth of 260 TIE-1 During Photoheterotrophic Growth with Butyrate.

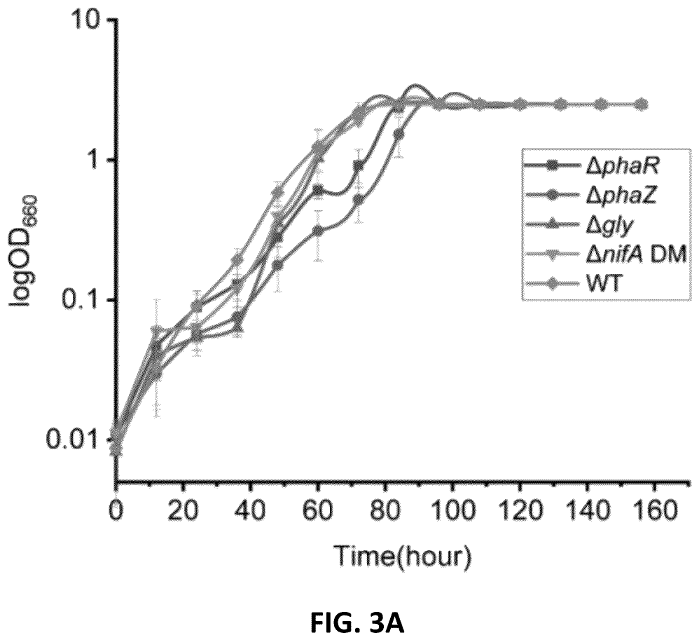

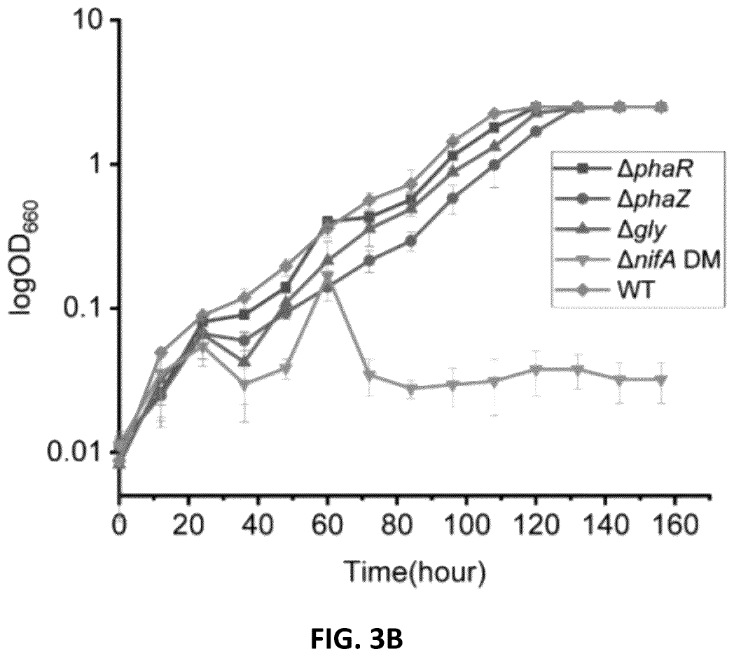

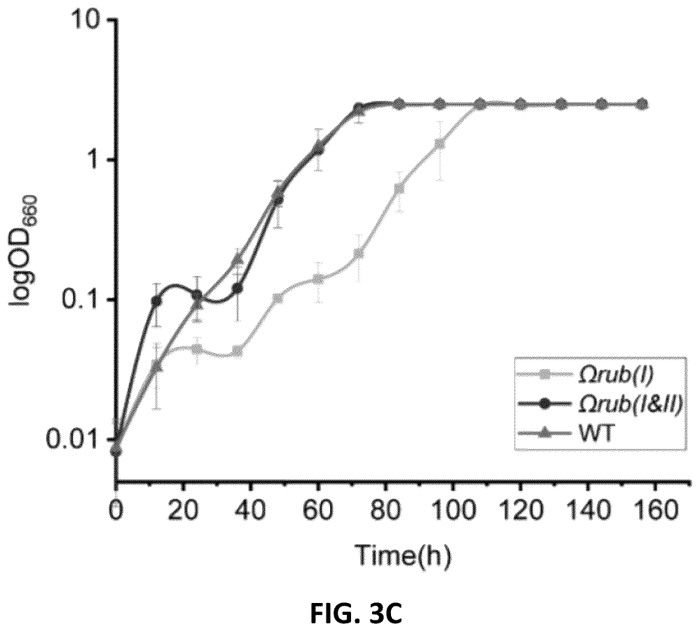

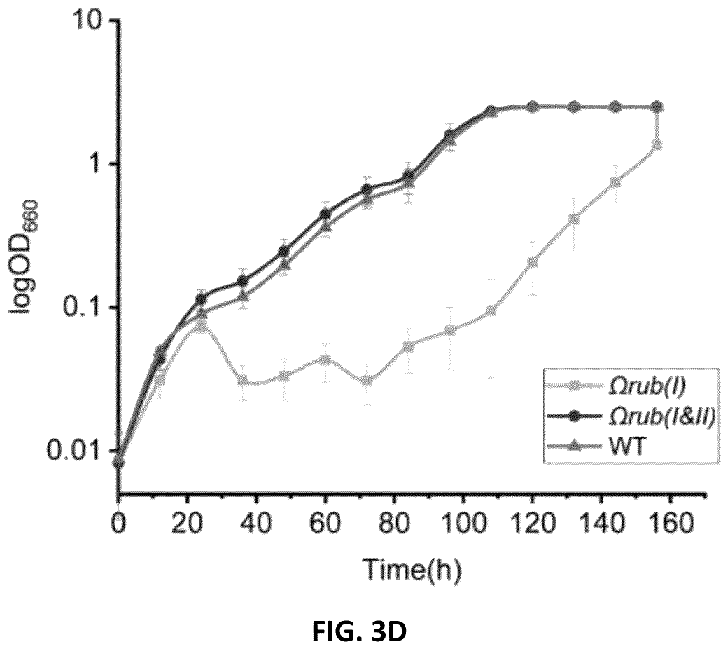

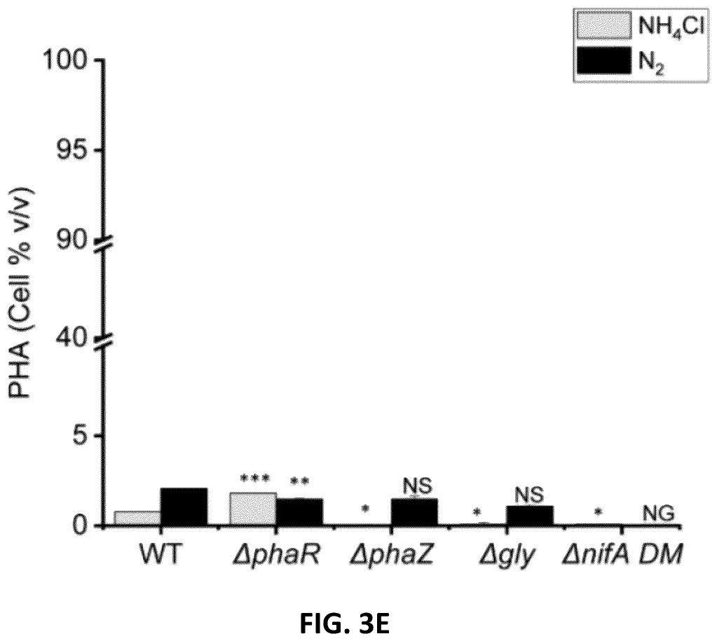

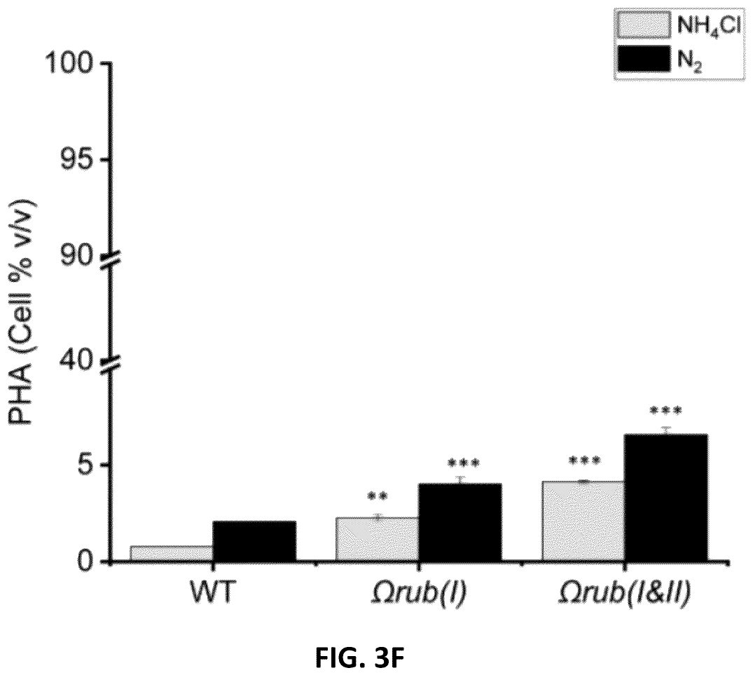

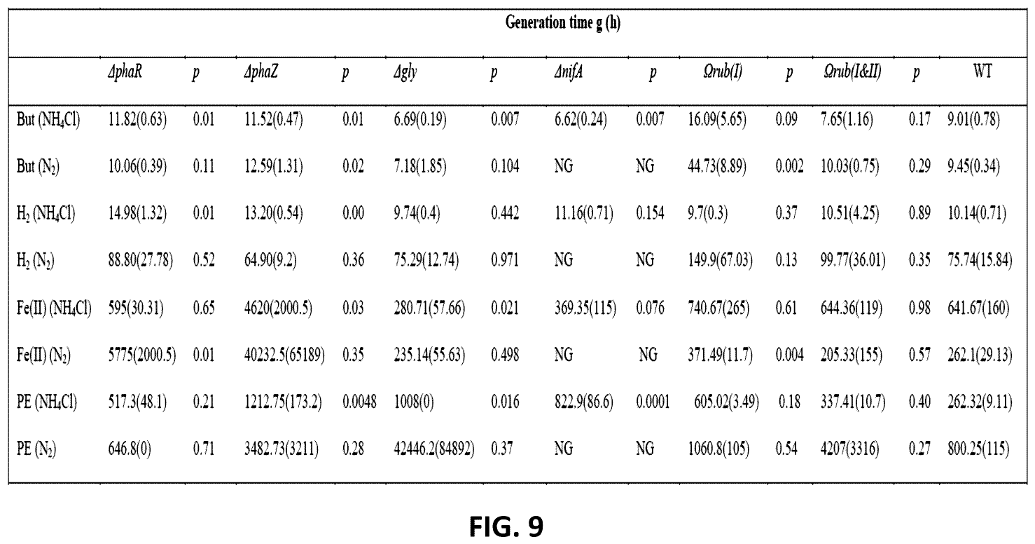

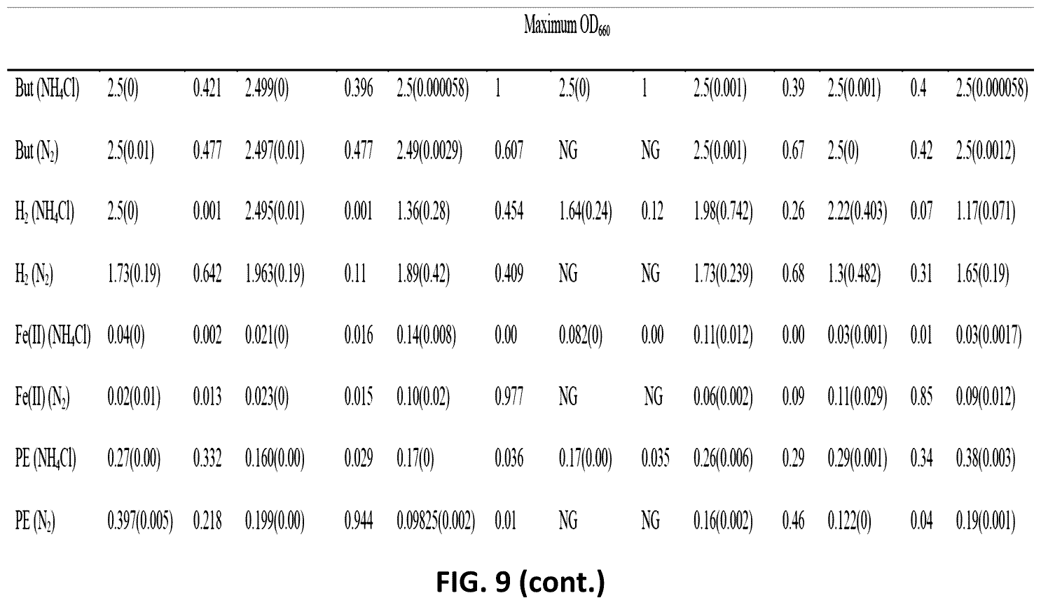

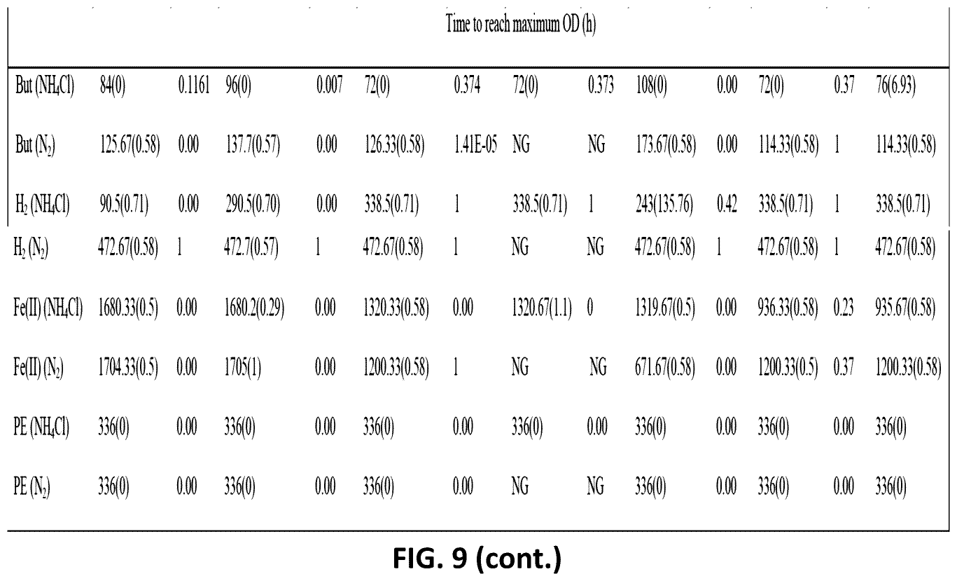

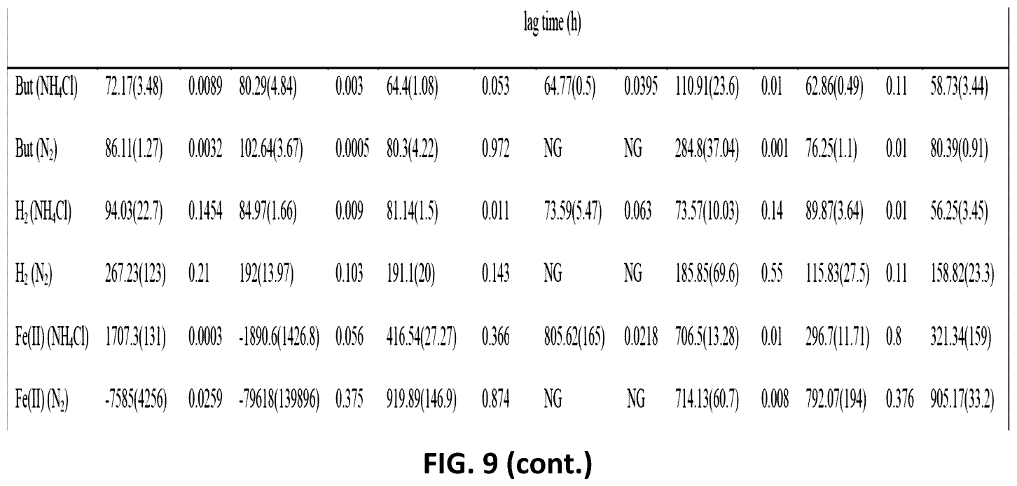

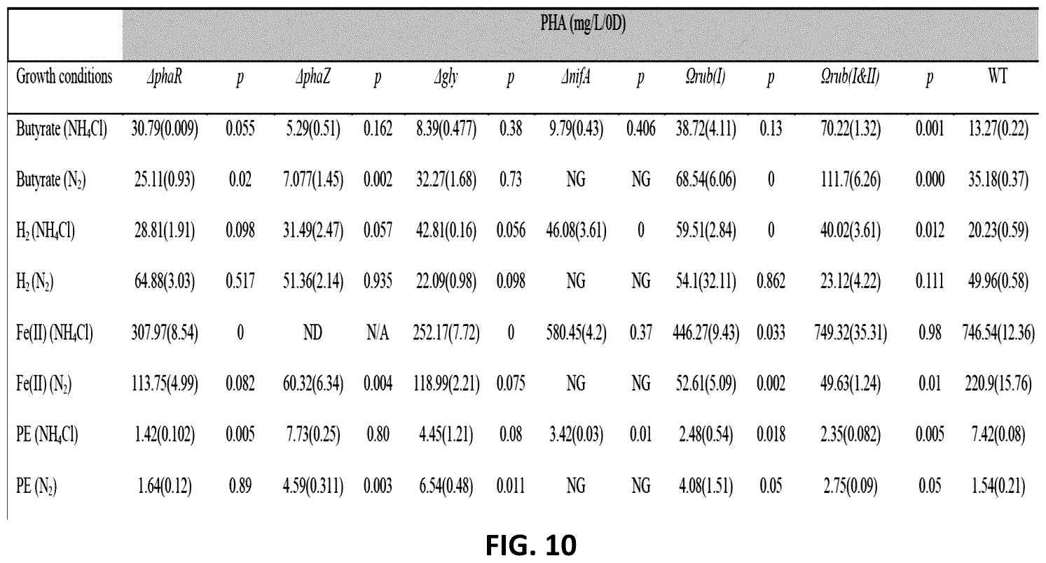

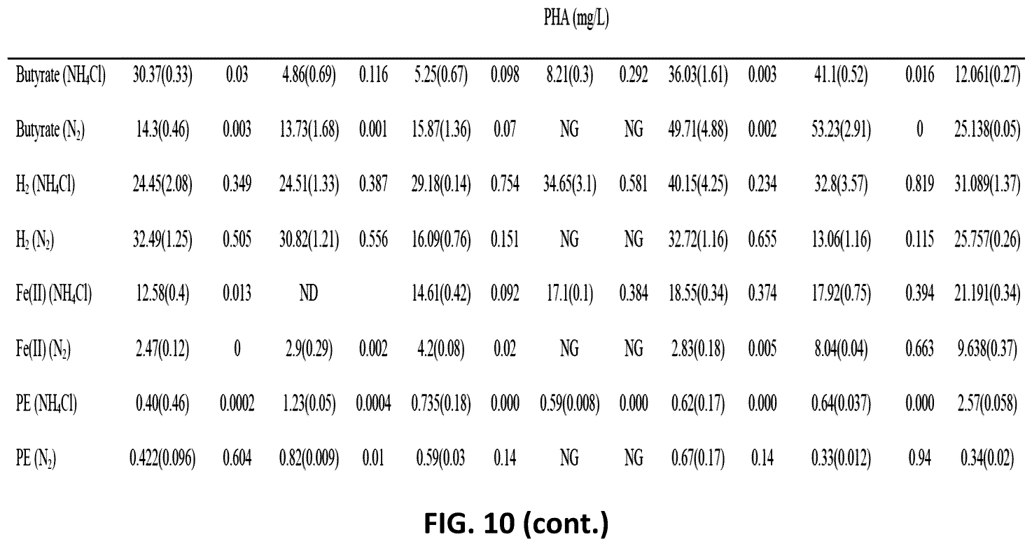

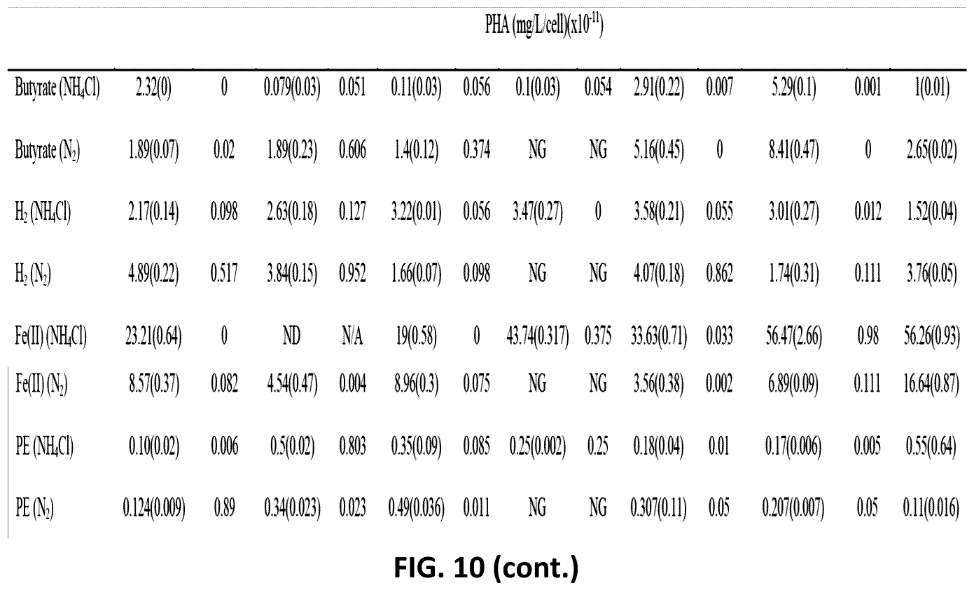

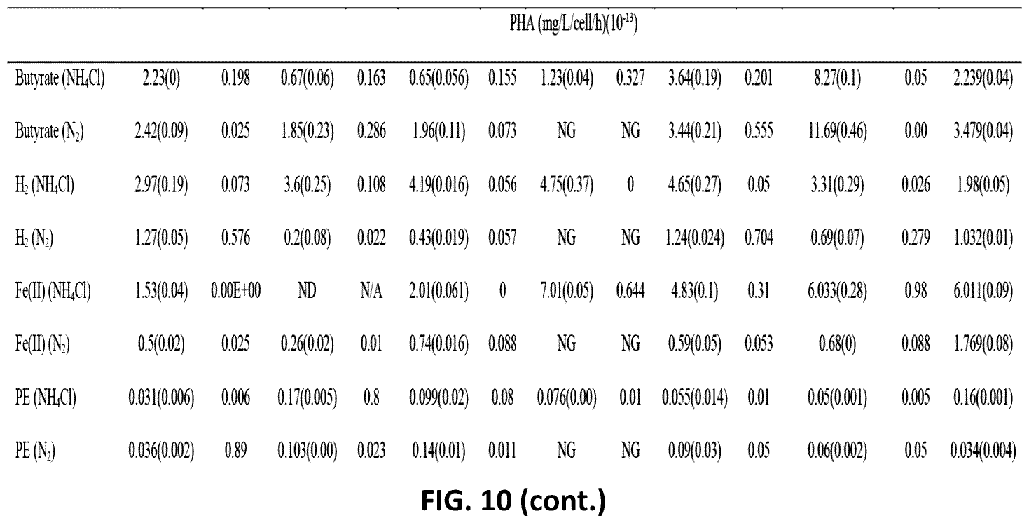

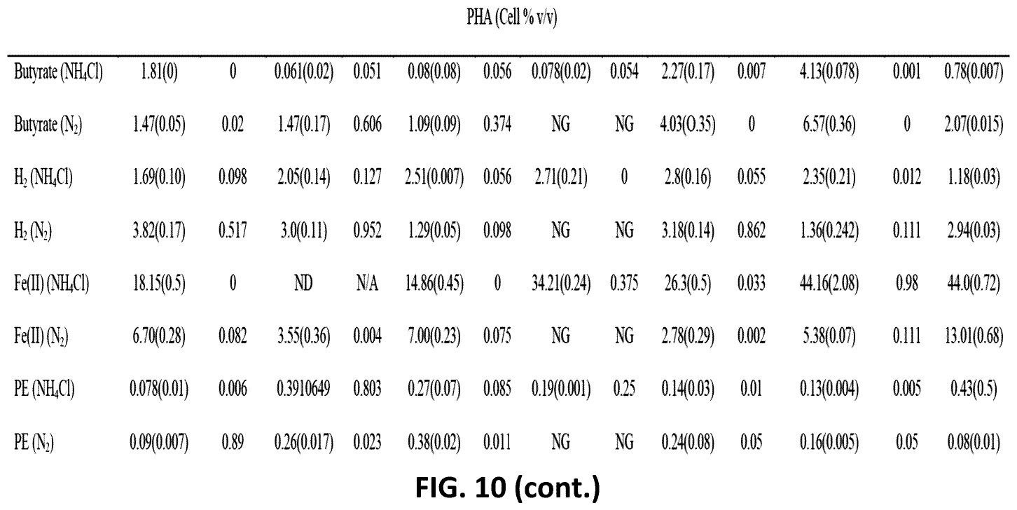

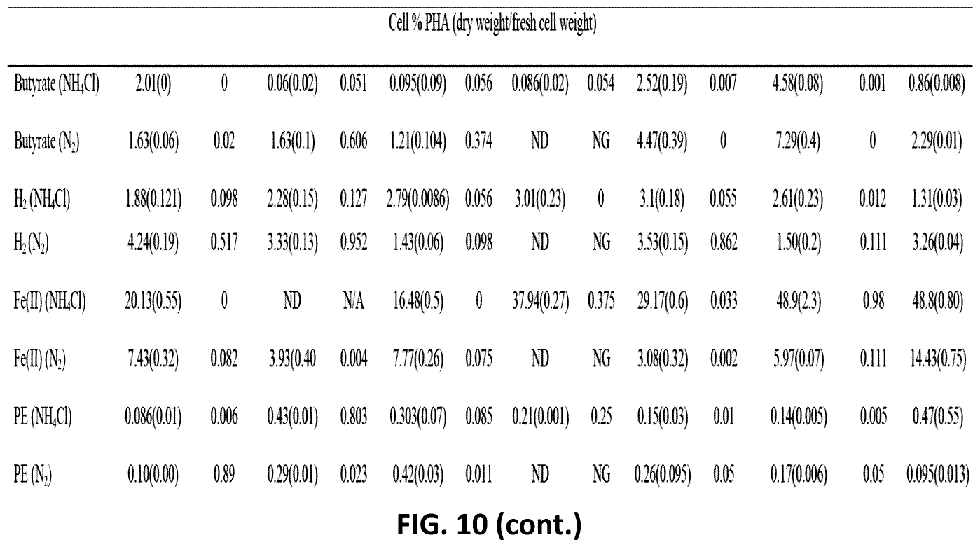

Because TIE-1 has previously exhibited high PHA production under anoxic photoheterotrophic conditions with butyrate, this growth condition using all the constructed strains was evaluated. Under these anoxic growth conditions, light is used as an energy source and butyrate is used both as electron and carbon source. For all growth conditions explored, the effect of nitrogen fixation by supplying N₂ gas (nitrogen-fixing) or ammonium chloride (NH₄Cl) salt (non-nitrogen-fixing) was determined. The results show that deleting phaR or phaZ increased the generation time of TIE-1 by around 3 hours compared to wild type when grown photoheterotrophically with butyrate under non-nitrogen fixing conditions (FIGS. 10 and 3A). However, a slightly shorter generation time (3 hours shorter than wild type) was obtained from the Δgly and the ΔnifA under these conditions. Under nitrogen-fixing conditions, the ΔphaZ mutant continued to show a growth defect observed as a longer generation time (3 hours longer than wild type), whereas the ΔphaR and Δgly mutants showed the same generation time as wild type. As expected, ΔnifA was not able to grow under nitrogen-fixing conditions (FIGS. 9 and 3B). The growth of the engineered RuBisCo strains Ωrub(I) and Ωrub(I&II) under the same photoheterotrophic growth condition with butyrate was compared. The engineered strains carrying only the overexpressed RuBisCo gene form I (Ωrub(I)) has a growth defect under both nitrogen-fixing and non-nitrogen fixing conditions (FIGS. 3C and 3D). This defect seems to be more noticeable under nitrogen-fixing growth. The Ωrub(I) strains have an ˜2-fold longer lag time than wild type when grown photoheterotrophically with butyrate under non-nitrogen fixing conditions, and ˜5-times longer generation time under nitrogen-fixing conditions (FIG. 9, 3C, and 3D). The engineered strain Ωrub(I&II) carrying overexpressed RuBisCo form I and II genes did not show any significant difference in growth compared to wild type when grown under non-nitrogen fixing or under nitrogen-fixing conditions (FIG. 9, 3C, and 3D).

Deletion of the phaR Gene or Overexpression of the RuBisCo form I and II Increases PHA 288 Production in TIE-1 Under Photoheterotrophic Growth with Butyrate.

Deletion of the phaR (regulator gene) increased PHA production (mg/L/Cell and cell % v/v) ˜2-fold compared to wild type under non-nitrogen or nitrogen-fixing conditions (FIGS. 3E and 10). Deletion of the phaZ, gly, or nifA genes resulted an overall decrease of

PHA production (mg/L/Cell and cell % v/v) of ˜10-fold compared to wild type under growth under non-nitrogen fixing conditions. These deletions did not seem to affect PHA production when compared to wild type under nitrogen-fixing conditions. PHA from the ΔnifA grown under nitrogen-fixing conditions were not detected as it did not show any growth. Except for ΔphaR, all strains exhibited a consistent increase in PHA production during nitrogen-fixing growth in comparison to growth under non-nitrogen fixing conditions.

When grown photoheterotrophically with butyrate, the two strains harboring the Ωrub(I) and Ωrub(I&II) demonstrated increased PHA production regardless of the nitrogen source. Under non-nitrogen fixing conditions, the Ωrub(I) strain had 2-fold higher PHA production (mg/L/Cell and cell % v/v) than wild type, while the Ωrub(I&II) exhibited a 5-fold increase. This increase in PHA was also observed under nitrogen-fixing conditions where an increase of 1.9-fold was observed from the Ωrub(I) and 3-fold from the Ωrub(I&II) (FIGS. 10 and 3F). Similar to the deletion mutants, an overall increase in PHA production was observed from the engineered and wild type strains under nitrogen-fixing vs. non-nitrogen fixing conditions (FIG. 3F).

Deletion of the phaR, phaZ, or Overexpression of RuBisCo form I and II Increased the Final Optical Density of TIE-1 Under Photoautotrophic Growth with H2 Under Non-Nitrogen Fixing Conditions.

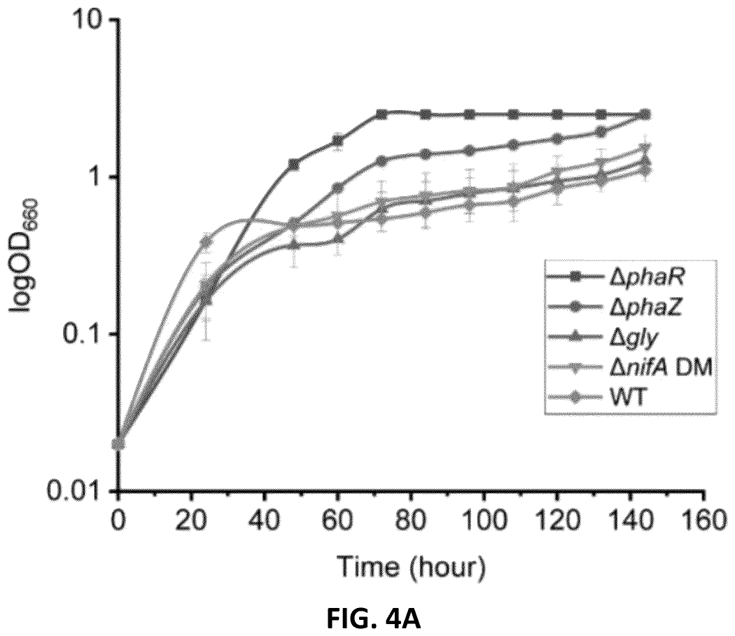

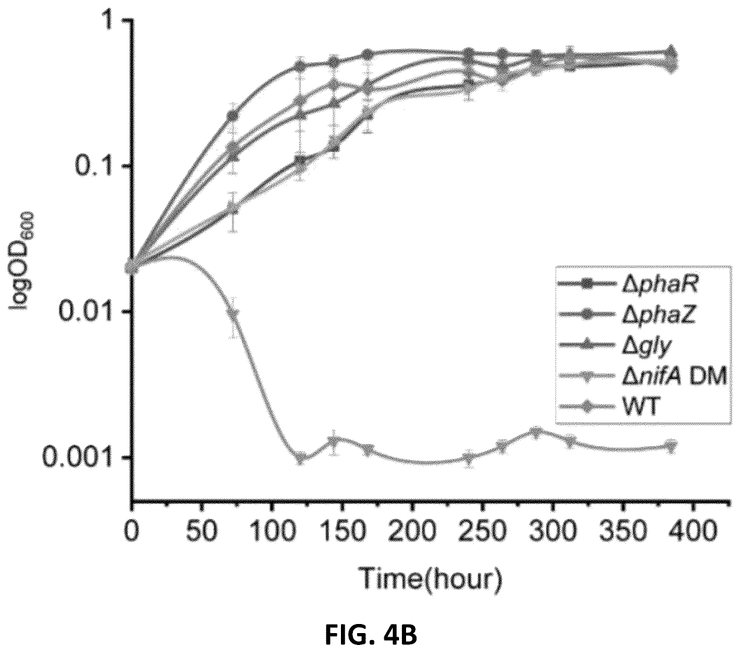

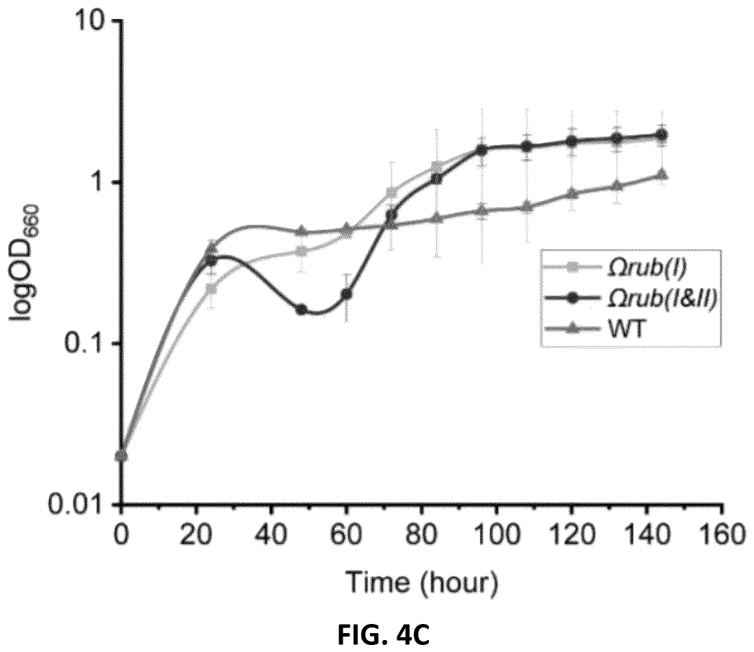

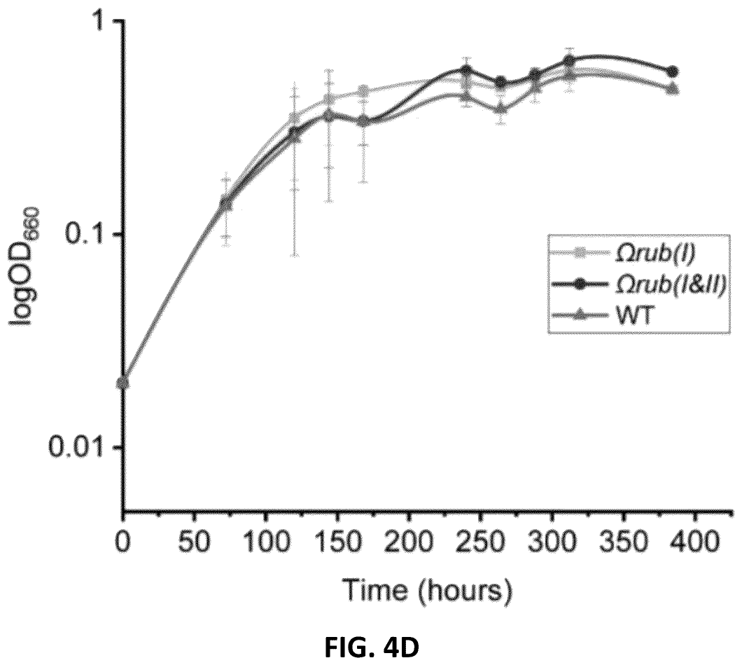

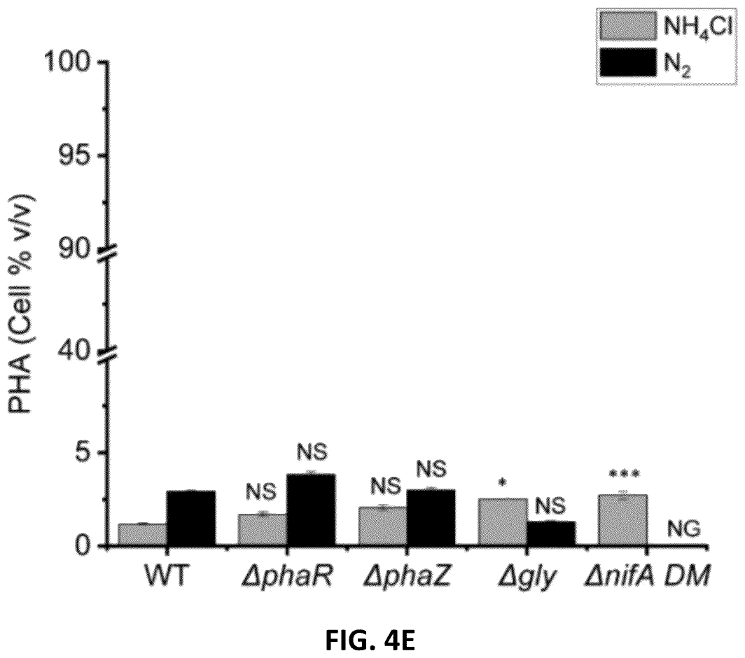

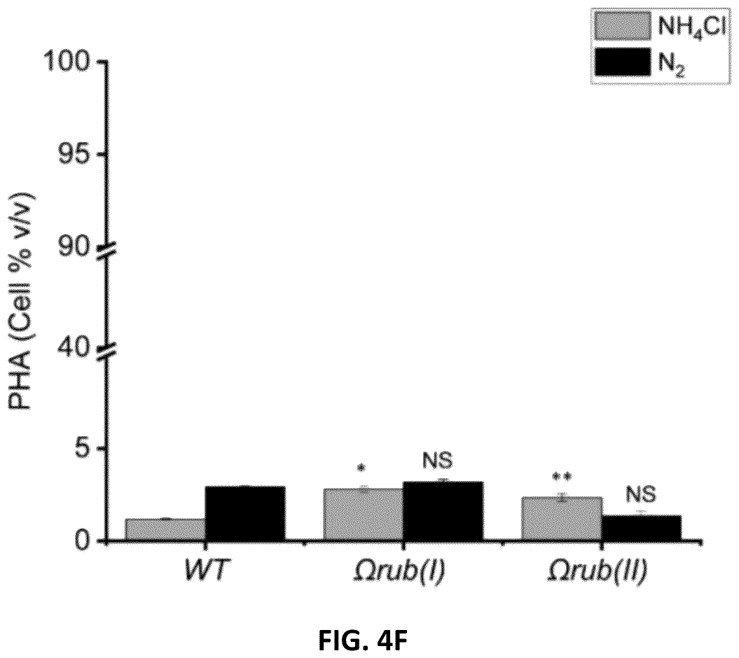

Achieving production from a low-cost and abundant carbon source is one of the most impactful pathways for bioplastic production. Accordingly, the growth of all the strains were tested under photoautotrophic conditions using H₂ as an electron source and CO₂ as a carbon source in a freshwater basal liquid medium. Light is used as the energy source as TIE-1 is a photosynthetic bacterium. FIGS. 4A and 9 show that although ΔphaR and ΔphaZ mutants showed a slightly longer generation time (˜3 hours slower), they reached the maximum OD faster than wild type when grown with H₂ under non-nitrogen fixing conditions. ΔphaR reached the maximum OD, 3.7 times faster than wild type whereas the ΔphaZ mutant reached maximum OD almost 50 hours earlier than wild type. Δgly and ΔnifA showed similar growth patterns as wild type under growth with H₂ and non-nitrogen fixing conditions (FIG. 4A). No significant difference was observed under nitrogen-fixing conditions with hydrogen from all the mutants except the ΔnifA which showed no growth as shown in FIGS. 9 and 4B.

Although the strains Ωrub(I&II) showed an extended lag time (1.6 slower than wild type), they reached a higher final OD (0.52 higher) than wild type (FIG. 9) when grown with H₂ and under non-nitrogen fixing conditions. The Ωrub(I) did not show any significant growth difference compared to wild type (FIGS. 9 and 4E). No difference was observed between the two engineered strains when grown with H₂ under nitrogen-fixing conditions (FIGS. 9 and 4D).

Deletion of the Glycogen Synthase (gly), nifA Genes, or Overexpression of RuBisCo form I or I&II Increased PHA Production Under Photoautotrophic Growth with H2 Under Non-Nitrogen Fixing Conditions.

PHA production (mg/L/Cell and cell % v/v) from all the constructed strains were tested under photoautotrophic growth conditions with H₂. The mutants ΔphaR and ΔphaZ did not show any significant difference in PHA production compared to wild type under growth with H₂ regardless of the nitrogen source. The Δgly and the ΔnifA strains showed a 2-fold increase in PHA production when grown with H₂ under non-nitrogen fixing conditions (FIGS. 10 and 4E). The deletion of phaR or phaZ did not affect PHA production compared to wild type. However, deletion of glycogen synthase caused a 50% loss of PHA production under nitrogen-fixing conditions. No PHA was produced from the ΔnifA during growth under nitrogen-fixing conditions due to its inability to grow. Unlike growth with butyrate, switching from non-nitrogen to nitrogen-fixing conditions increased PHA production only in wild type TIE-1 but not in the mutants. The mutants showed lower PHA production compared to wild type under nitrogen-fixing conditions with H₂ as an electron source.

Similar to growth with butyrate, both engineered Ωrub(I) and Ωrub(I&II) strains showed an increase in PHA production (mg/L/Cell and cell % v/v) when grown with H2 under non-nitrogen fixing conditions. PHA production was nearly double in both engineered strains compared to wild type, (FIGS. 10 and 4F). Switching to nitrogen-fixing conditions with H₂ did not affect PHA production (mg/L/Cell and cell % v/v) of the two engineered strains Ωrub(I) and Ωrub(I&II) when compared to wild type (FIGS. 10 and 4F). In contrast, a decrease in PHA production was observed from the Ωrub(I&II) under these conditions (FIGS. 10 and 4F).

Deletion of the phaR and phaZ Genes Impaired the Ability of TIE-1 to Oxidize Fe(II)

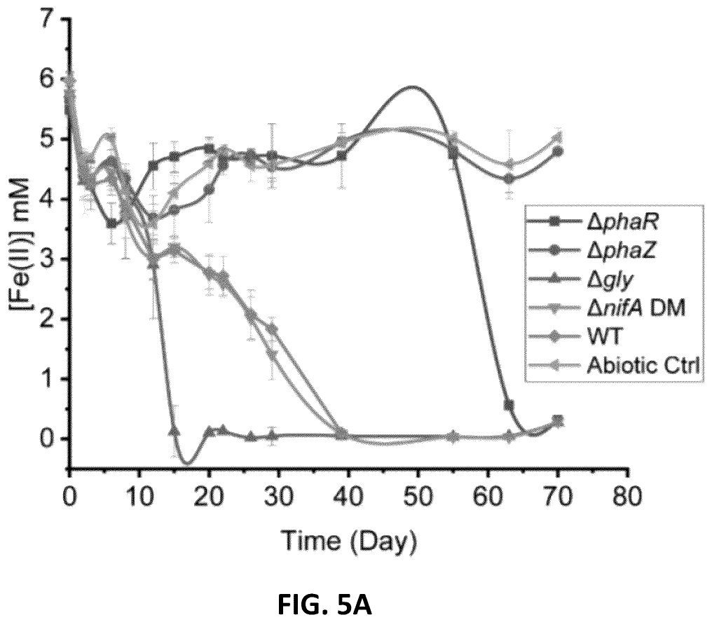

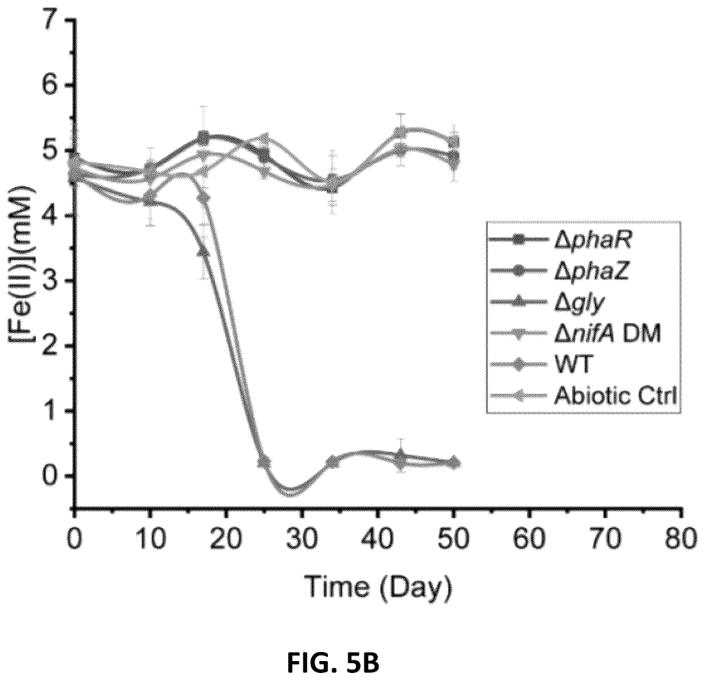

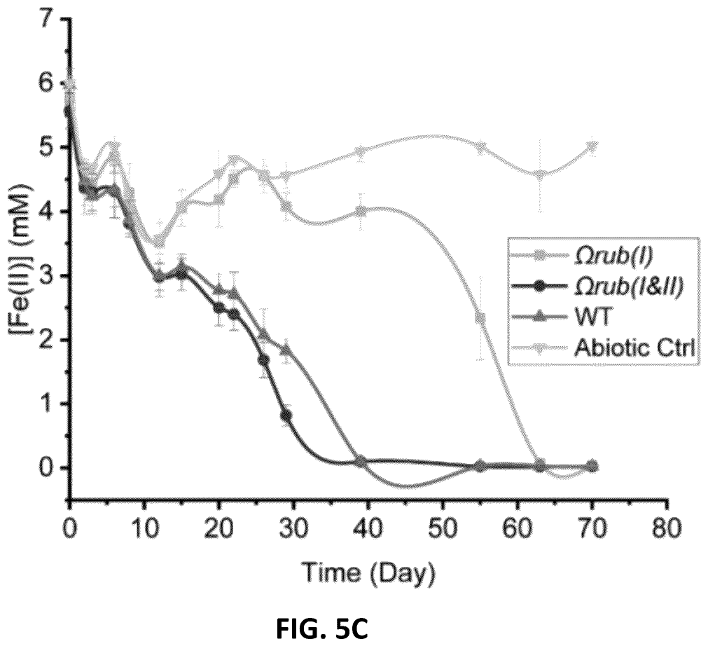

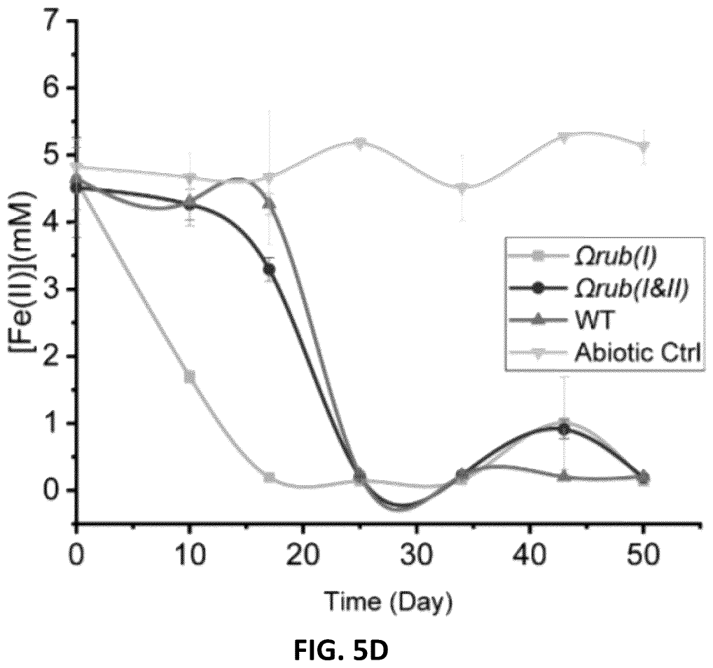

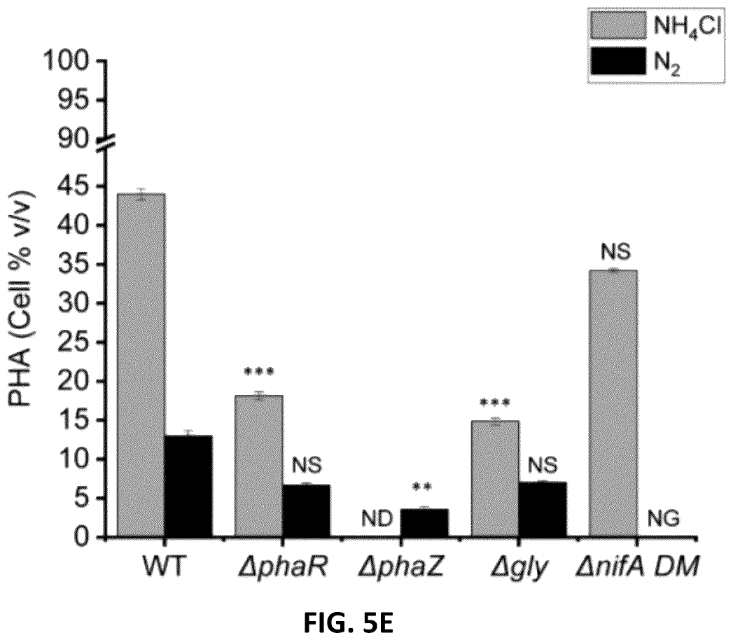

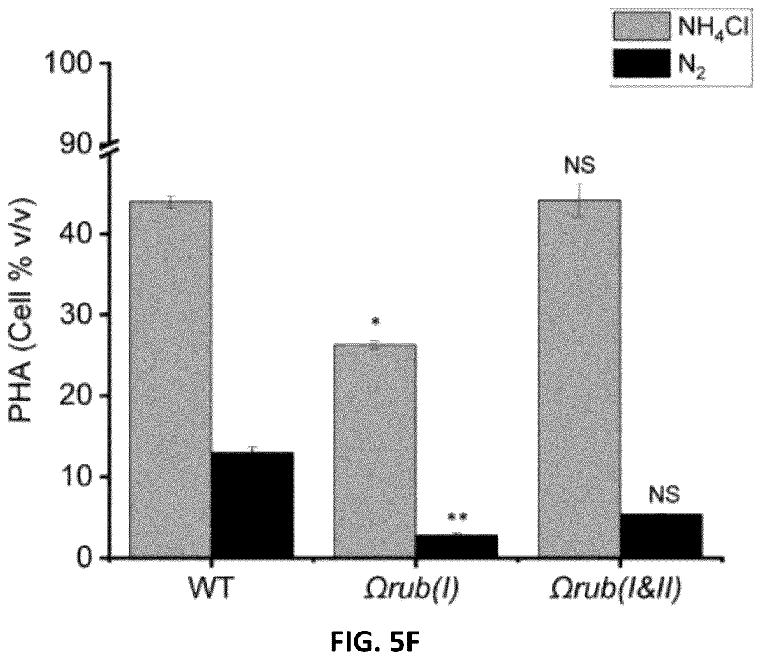

TIE-1 can use electrons produced by the oxidation of Fe(II) for photoautotrophy (photoferrotrophy). Under these growth conditions, the main carbon source is CO₂, and the energy source is light. The growth of all the strains that were constructed under photoferrotrophic growth conditions were tested. All the strains were first pre-grown in H2 to allow expression of the genes involved in Fe(II) oxidation in TIE-1. A defect in the ability of TIE-1 to oxidize Fe(II) when the phaZ or phaR genes were deleted (FIGS. 5A and 5B) was observed. Under non-nitrogen fixing conditions, the phaZ mutant was not able to oxidize Fe(II) even after 50 days of growth. A significant delay in Fe(II) oxidation was observed in the phaR mutant under non-nitrogen fixing conditions. ΔphaR was able to fully oxidize Fe(II) only after 65 days versus 40 days for wild type. In contrast, Δgly showed faster Fe(II) oxidation ability which occurred after 15 days of growth as opposed to ˜40 days for wild type when grown under non-nitrogen fixing conditions. The ΔnifA showed a similar iron oxidation pattern as wild type under non-nitrogen fixing conditions (FIG. 5A). Under nitrogen-fixing conditions, the oxidation ability of Δgly was similar to wild type. As expected, no Fe(II) oxidation occurred from ΔnifA as the strain was not able to grow under nitrogen-fixing conditions. Overexpression of RuBisCo form I appears to delay the ability of TIE-1 to oxidize Fe(II) under non-nitrogen fixing conditions. However, when RuBisCo form I and II are both overexpressed, the strain oxidizes Fe(II) at the same time as wild type (˜40 days) (FIG. 5C). During nitrogen fixation, the Ωrub(I) showed faster Fe(II) oxidation compared to wild type, whereas the Ωrub(I&II) initiated Fe(II) oxidation at the same time as wild type (FIG. 5D).

Deletion of the Various Genes or Overexpression of RuBisCo form I and II did not Improve 384 PHA Production in TIE-1 Under Photoferrotrophy.

To test PHA production obtained from growth during photoferrotrophy, samples were collected right after complete Fe(II) oxidation occurred. Lower PHA production (mg/L/Cell and cell % v/v) was observed across all the mutants under growth with Fe(II) and non-nitrogen fixing conditions compared to wild type (FIGS. 10 and 5E). Among all the mutants, the ΔnifA had the highest PHA production.

ΔphaR and Δgly showed the lowest production, about half that observed in wild type when grown under non-nitrogen fixing conditions. No PHA was detected from the ΔphaZ mutant from the non-nitrogen fixing conditions which is linked to its inability to oxidize Fe(II). Like growth under non-nitrogen fixing conditions, PHA production for all the mutant strains during nitrogen-fixing conditions was overall lower than that obtained from wild type (FIGS. 5E and 5F). PHA production (mg/L/Cell and cell % v/v) obtained under nitrogen-fixing conditions was ˜3× smaller than under non-nitrogen fixing conditions for wild type. ΔphaR and Δgly, showed low PHA production compared to wild type when grown under nitrogen-fixing conditions. PHA production obtained from these two mutants was almost half that of wild type. Deleting the phaZ gene of TIE-1 decreased its production to about ¼ of that obtained from wild type under nitrogen-fixing conditions (FIGS. 10 and 5E). No PHA was obtained from ΔnifA when grown under nitrogen-fixing conditions because this strain is incapable of growth under such conditions.

Although a decrease in PHA production of about half was observed from the single engineered Ωrub(I) strain under Fe(II) under non-nitrogen fixing growth conditions, a similar PHA production wild type was obtained from the engineered strain carrying both RuBisCo form I and II genes (FIG. 10 and FIG. 5F). PHA production obtained from the Ωrub(I) was about half of that obtained from wild type under growth with Fe(II) under non-nitrogen fixing conditions. Like the trend obtained from the mutants, a decrease in PHA production was observed in the RuBisCo overexpressing strains under nitrogen-fixing conditions compared to the growth under non-nitrogen fixing conditions. PHA production obtained from the Ωrub(I) was ˜4× less than what was obtained from wild type under nitrogen-fixing conditions. PHA production obtained from the Ωrub(I&II) strain dropped to 2.4 times less than wild type under the same conditions.

Growth Under Photoelectrotrophy Under Non-Nitrogen Fixing Conditions Showed Increased Electron Uptake from the Engineered RuBisCo Strains.

In addition to its ability to grow autotrophically using hydrogen and Fe(II) as electron sources, TIE-1 can uptake electrons directly from poised electrodes. The cells were grown in bioelectrochemical reactors as described in the Materials and Methods section with freshwater media under nitrogen-fixing or non-nitrogen fixing conditions. All mutant strains except ΔphaR showed longer generation time compared to wild type when grown under non-nitrogen fixing conditions (FIG. 9). ΔphaZ and Δgly exhibited significantly slower growth, as evidenced by a generation time ˜4× longer than wild type. ΔnifA similarly exhibited an extended generation time, ˜3× longer than that of wild type (FIG. 9). ΔphaR did not exhibit a significant difference in its generation time compared to wild type when grown under non-nitrogen fixing conditions. Ωrub(I) and Ωrub(I&II) did not show a defect in generation time compared to wild type under photoelectrotrophic non-nitrogen fixing conditions.

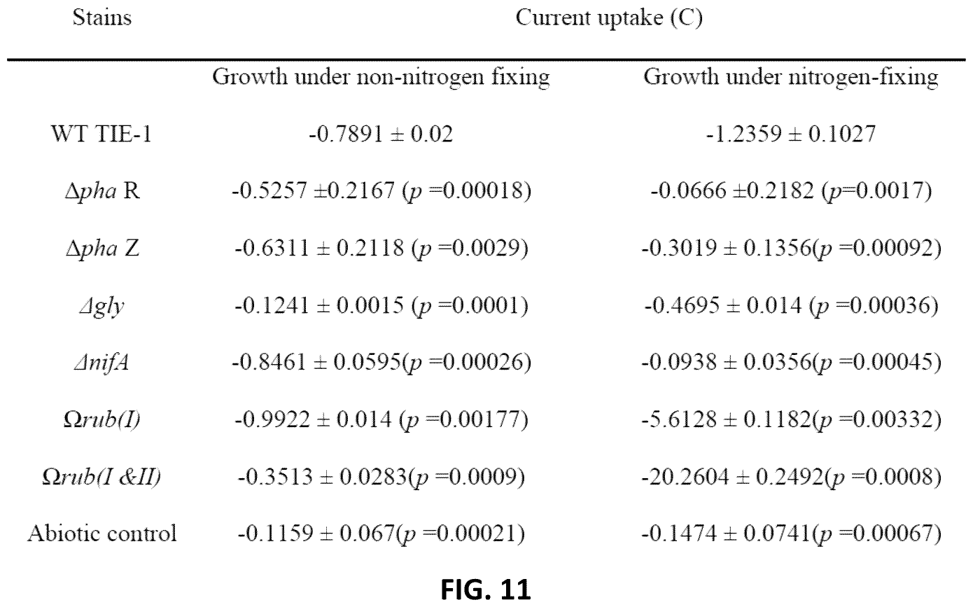

The final OD of the strains at the end of the fourteen-day incubation was measured. The final OD values were ˜½ lower than wild type from ΔphaZ, Δgly, and ΔnifA under non-nitrogen fixing growth conditions. ΔphaR as well as the engineered strains Ωrub(I) and Ωrub(I&II) had the same final OD as wild type (FIG. 9) when grown under non-nitrogen fixing conditions. Under photoelectrotrophic and nitrogen-fixing conditions, the generation time of ΔphaR, ΔphaZ, as well as Ωrub(I) and Ωrub(I&II) did not show any significant difference 436 compared to wild type (FIG. 7). The ΔnifA did not show growth as expected. The ability of each constructed strain to capture electrons from a poised electrode was assessed. Under non-nitrogen fixing conditions, ΔphaR, ΔphaZ, ΔnifA, Ωrub(I) did not show a significant difference in current uptake when compared to wild type. However, Δgly showed ˜6× lower current uptake, while Ωrub(I&II) showed a decrease of ˜2× in electron uptake when compared to wild type (FIG. 11).

Under nitrogen fixation conditions, electron uptake varied among strains. Notably, ΔphaR exhibited an 18× reduction compared to wild type. Additionally, both ΔphaZ and Δgly displayed 4- and 2.6× lower uptake, respectively. The ΔnifA, expectedly unable to grow under nitrogen fixation conditions, demonstrated an electron uptake similar to abiotic control. In contrast, Ωrub(I) and Ωrub(I&II) displayed 4.5 and 16× higher electron uptake than wild type, respectively (FIG. 11).

### Overexpressing the RuBisCo Form I and Form II of TIE-1 Increased its PHA Production Under Photoelectrotrophic Nitrogen Fixing Growth Conditions but not Under Non-Nitrogen Fixing Conditions.

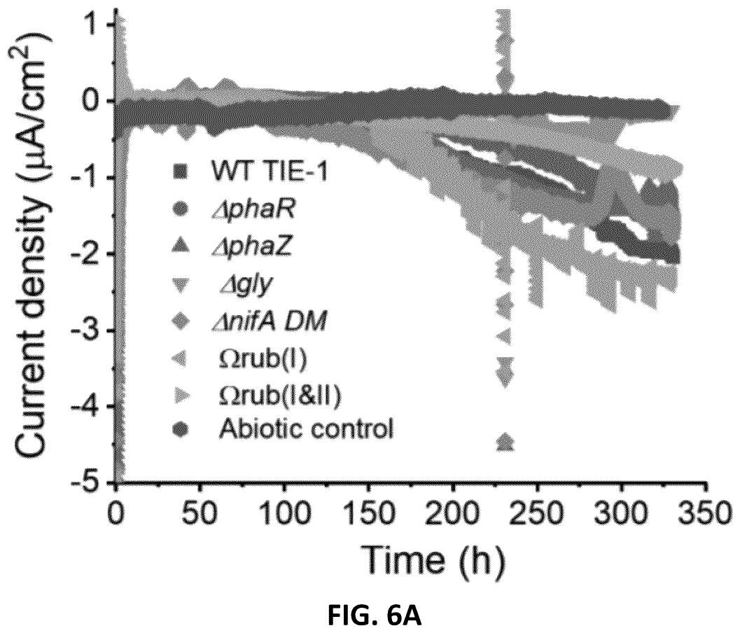

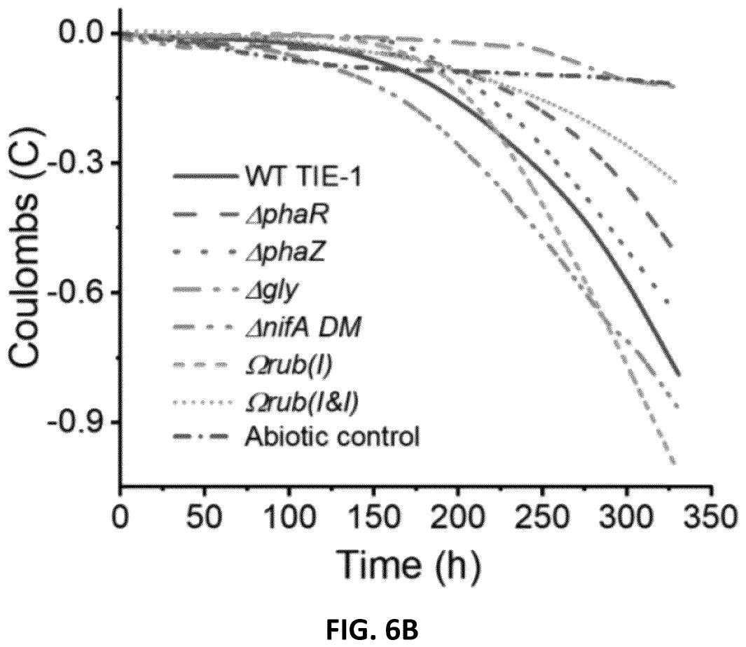

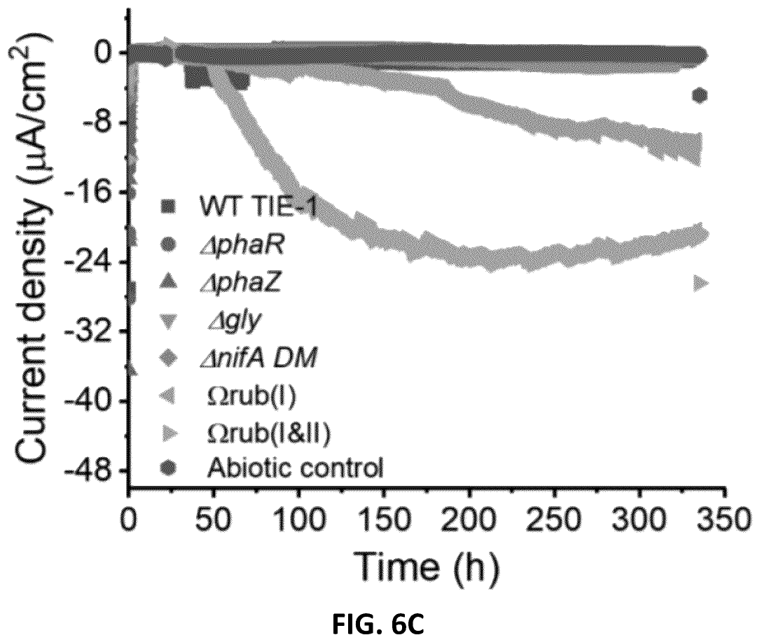

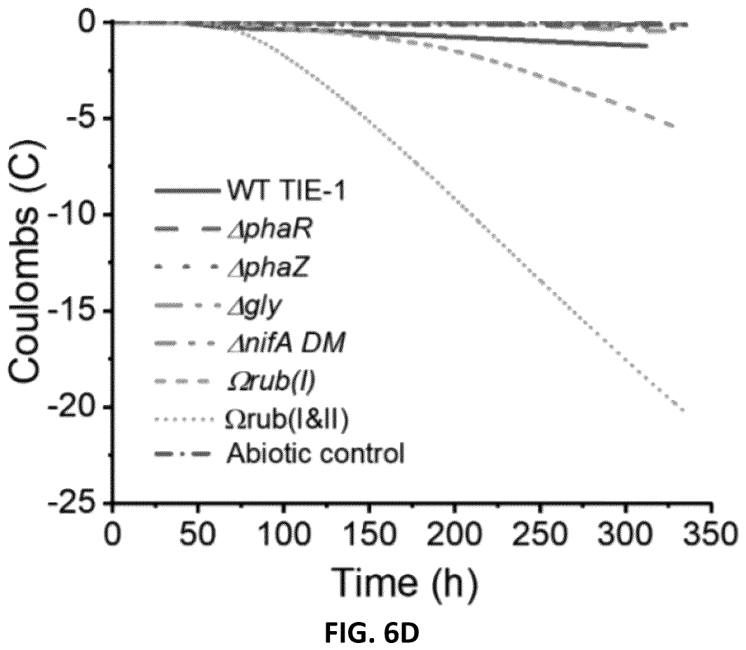

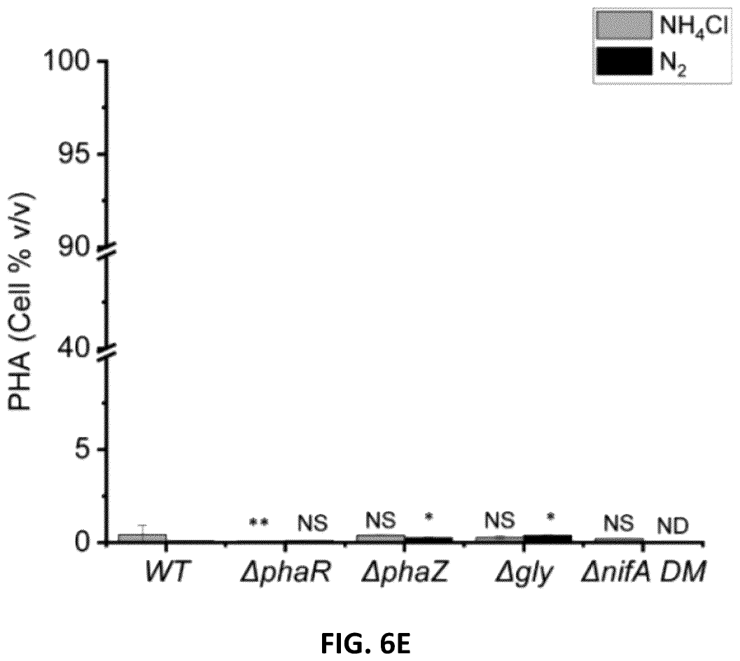

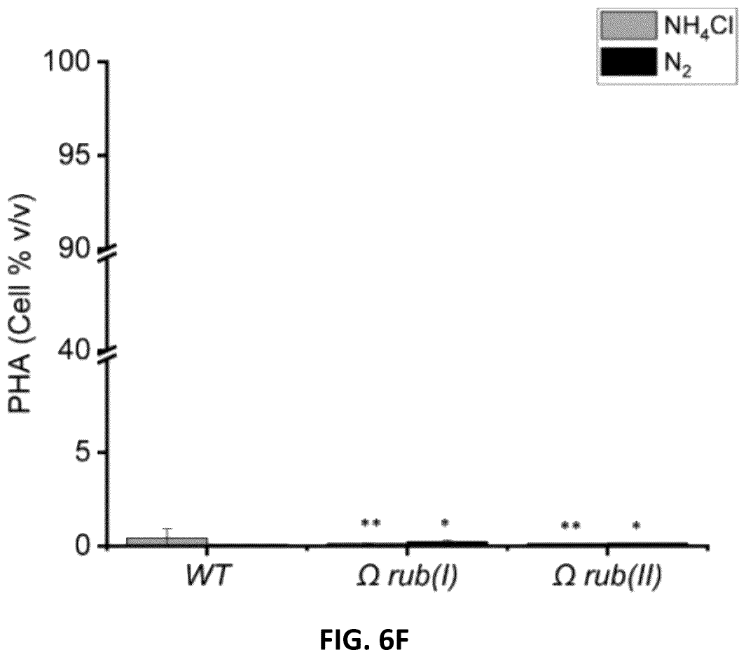

When grown under non-nitrogen fixing conditions, deletion of the phaR gene reduced PHA production (mg/L/Cell and cell % v/v) in TIE-1 with the lowest yield among all strains, accounting for just ⅕ of wild type as shown in FIGS. 10 and 6E. ΔphaZ, Δgly or ΔnifA did not show any difference in PHA production compared to wild type when grown photoelectrotrophically under non-nitrogen fixing conditions. However, overexpressing RuBisCo I or both form I and II in TIE-1 decreased PHA production to ˜3× compared to wild type TIE-1 under non-nitrogen fixing conditions (FIGS. 10 and 6F).

When grown under nitrogen-fixing conditions, it was observed that the phaR has a similar PHA production to wild type. However, PHA production increased by 3× in ΔphaZ and Δgly compared to wild type (FIGS. 10 and 6E). PHA production by Ωrub(I) and Ωrub(I&II) went 2-fold when compared to wild type. However, under nitrogen-fixing conditions PHA production obtained from Ωrub(I)=0.24% and Ωrub(I&II)=0.16% (cell % v/v) did not show statistically significant difference (p=0.21) when evaluated against each other.

### Materials and Methods

### Bacterial Strains, Media, and Growth Conditions

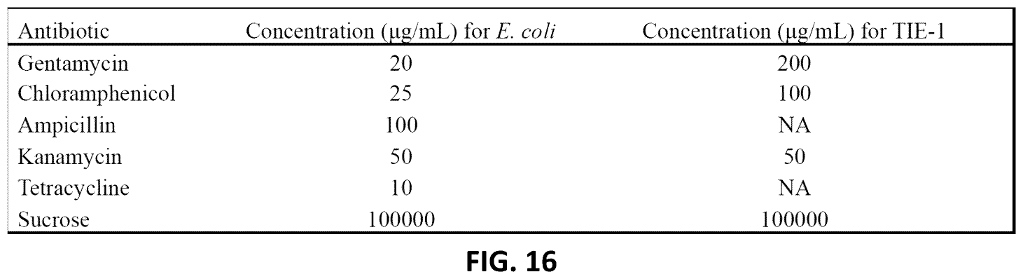

FIG. 7 lists all the strains used in the study. Lysogeny broth (LB) was used for growth of all E. coli strains at 37° C. Rhodopseudomonas palustris TIE-1 was grown in medium containing 3 g/L 602 yeast extract, 3 g/L peptone, 10 mM MOPS [3-N (morpholino) propanesulphonic acid] (pH 7.0), and 10 mM succinate (YPSMOPS) at 30° C. LB and YPSMOPS agar plates were prepared with the addition of 15 g/L agar. When needed, an antibiotic or sucrose was added as indicated in FIG. 16. All E. coli strains were grown on Lysogeny Broth (LB) at 37° C. FIG. 7 contains a list of the strains used in the study. FIG. 16 shows the concentration of antibiotics used as positive and negative selection components.

Anerobic growth of TIE-1 with hydrogen, Fe(II) or using poised electrodes were performed as previously described. Because, transitioning between heterotrophic to photoautotrophic growth conditions in H₂ and Fe(II) requires a metabolic shift in TIE-1, to obtain stable growth, cells were first pre-grown in yeast extract and then transferred into fresh water basal medium with H₂ for an additional pre-growth before the growth study. For growth under photoelectrotrophy, cell were grown with YP, washed three times with fresh water media and used directly to inoculate the reactors to reduce contamination. The bioelectrochemical systems were set up as previously described with the electrode modified to carbon felt (dimension 1×1×0.5 cm).

### Plasmid Construction

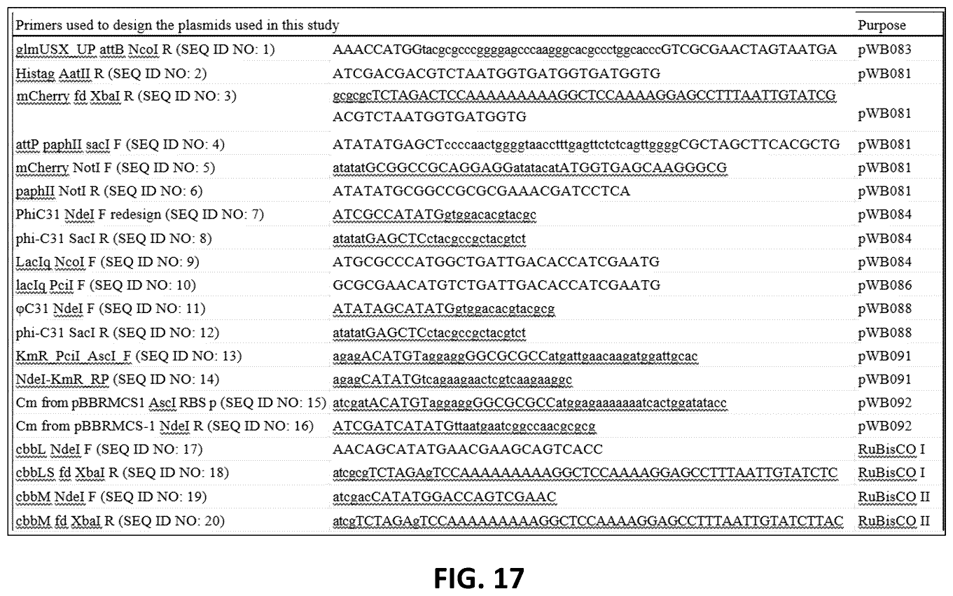

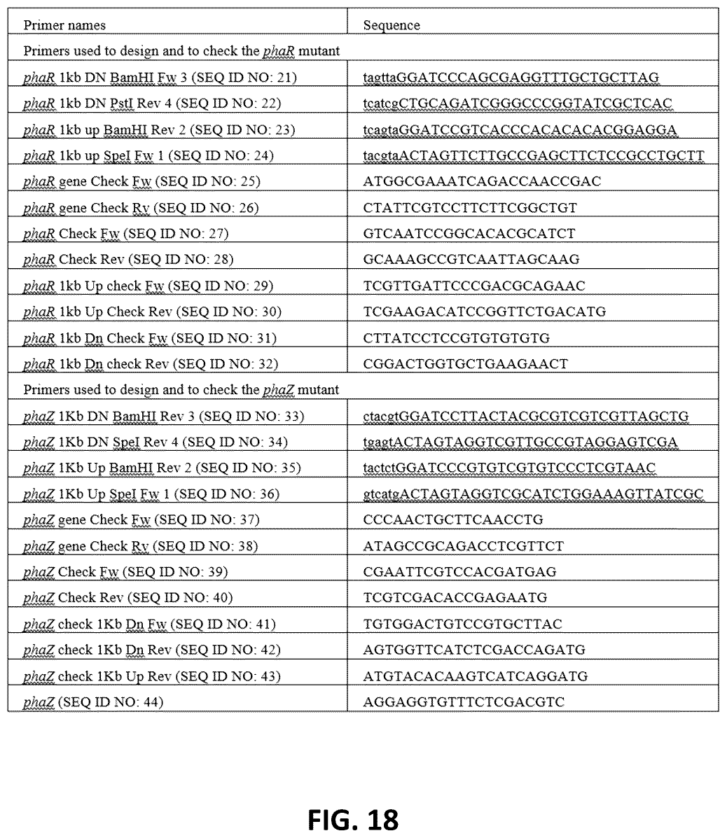

All the plasmids used in this Example are listed in FIG. 8. The kanamycin and chloramphenicol gene sequences were PCR amplified from pSRKKm and pSRKCm, respectively. All these antibiotic resistance marker genes were then cloned into pJQ200KS plasmid separately to replace the gentamicin resistance gene resulting in pWB091 and pWB092. All the primers used are listed in FIG. 17.

### φC31 Integrase Strain Construction

The φC31 attB and attP sequences are obtained from the previously published sequences. The attP sequence was cloned into pJQ200KS, resulting in pWB081. Then the PaphII-mCherry-fd cassette was cloned into pJQ200KS, resulting in pWB081. For the plasmid-based system, the φC31 integrase sequence was cloned into either plasmid pSRKGm or pWB081, resulting in pWB084 and pWB088. For the genome-based system, the φC31 integrase sequence was cloned to pAB314, resulting in pWB089. The attB sequence was also cloned into pAB314, resulting in pWB083.

Construction of Engineered TIE-1 Overexpressing RuBisCo form I and form II

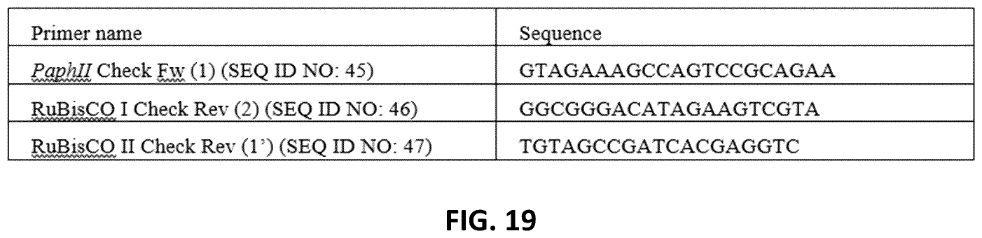

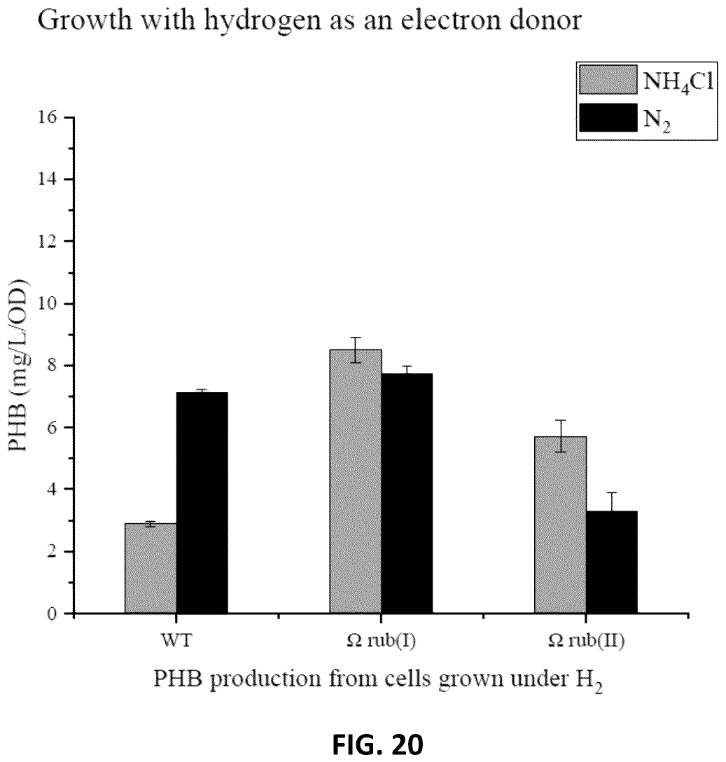

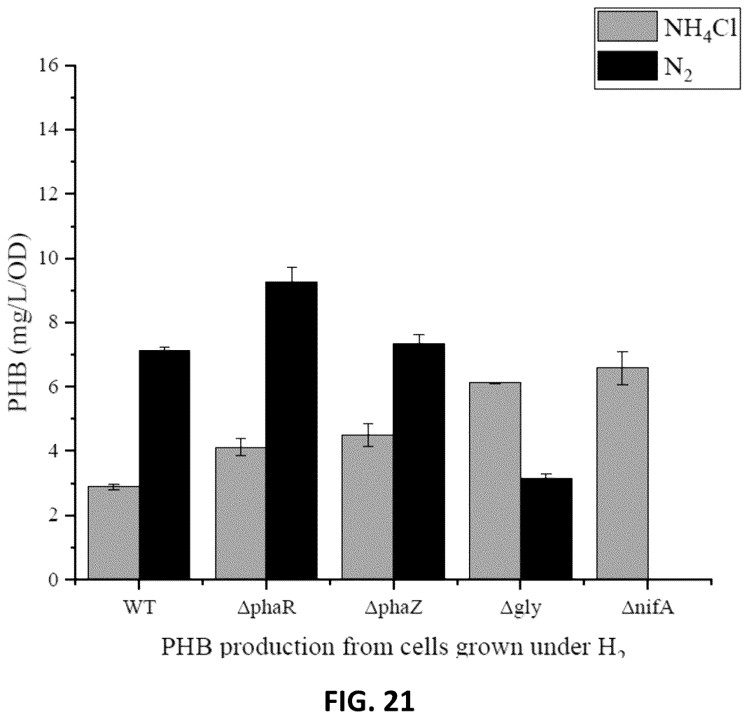

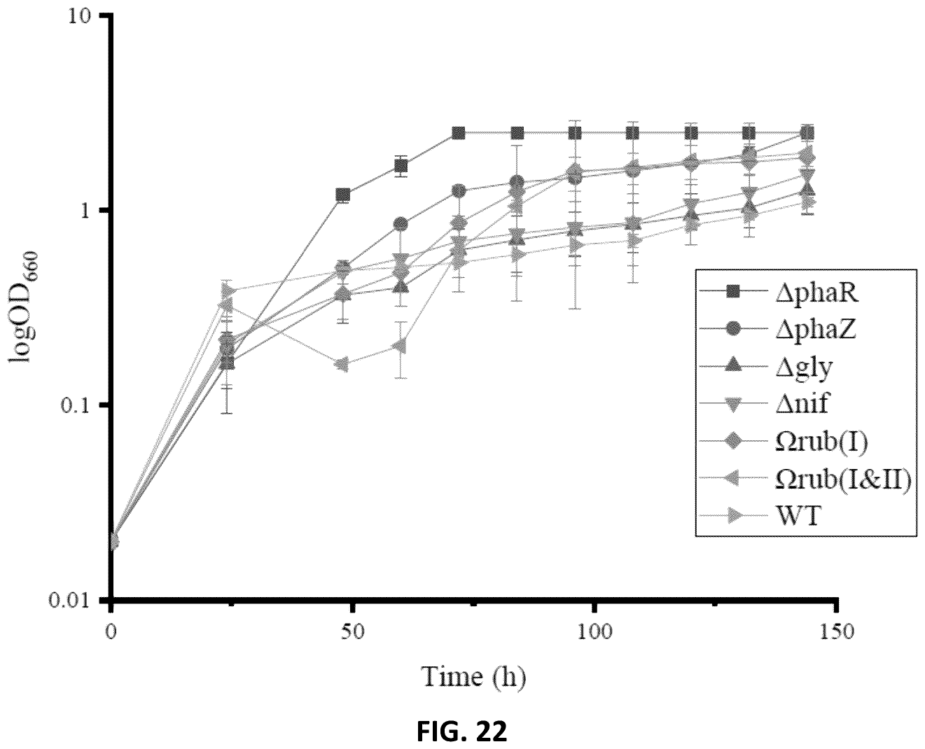

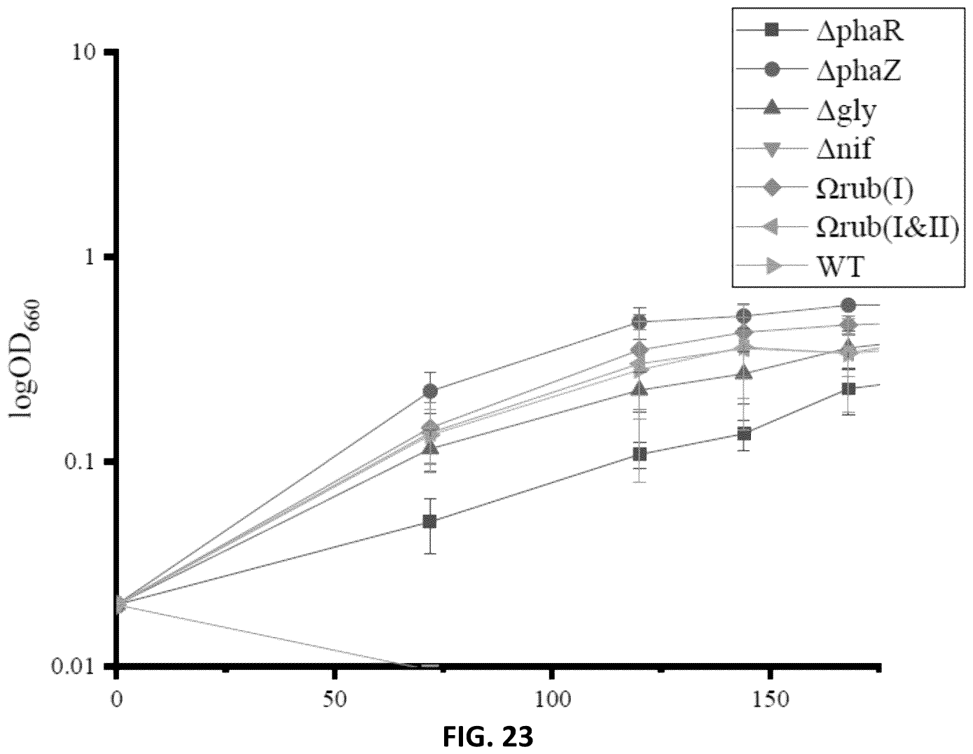

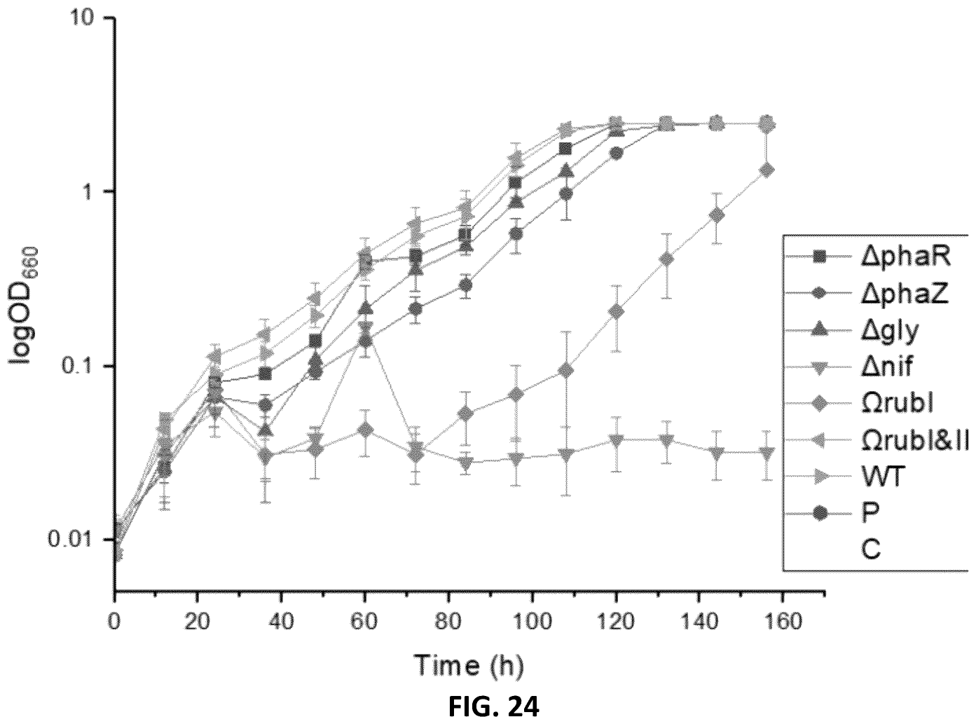

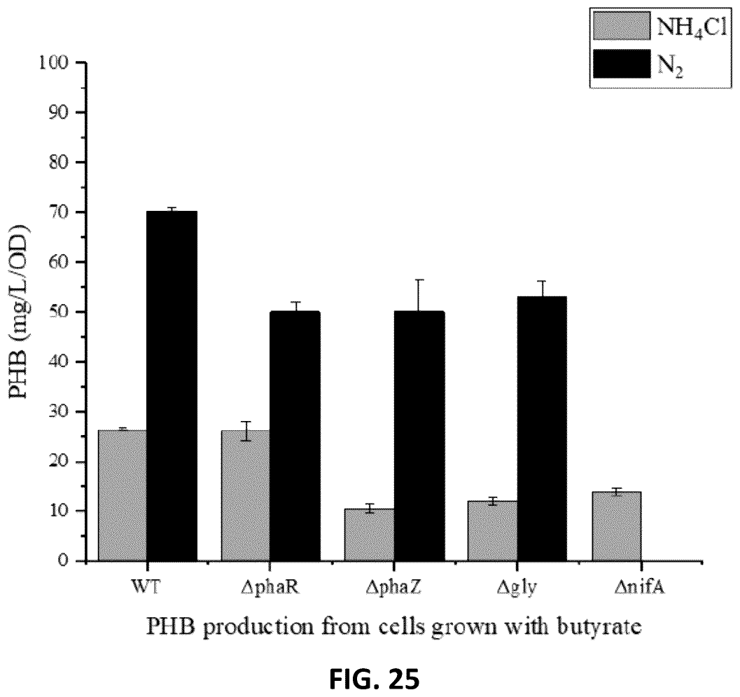

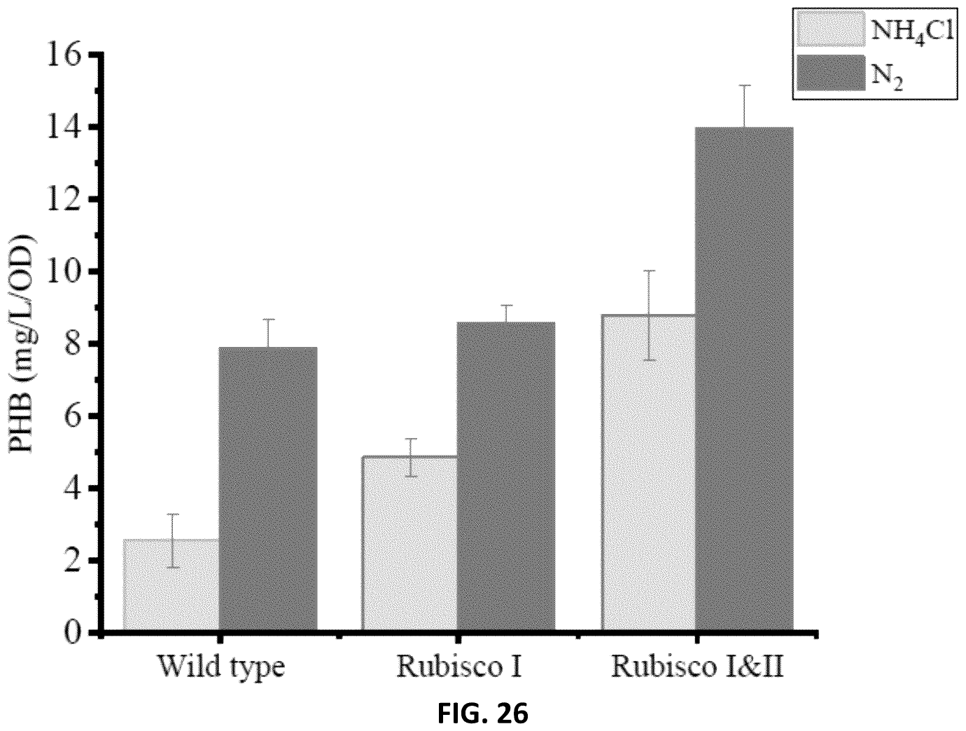

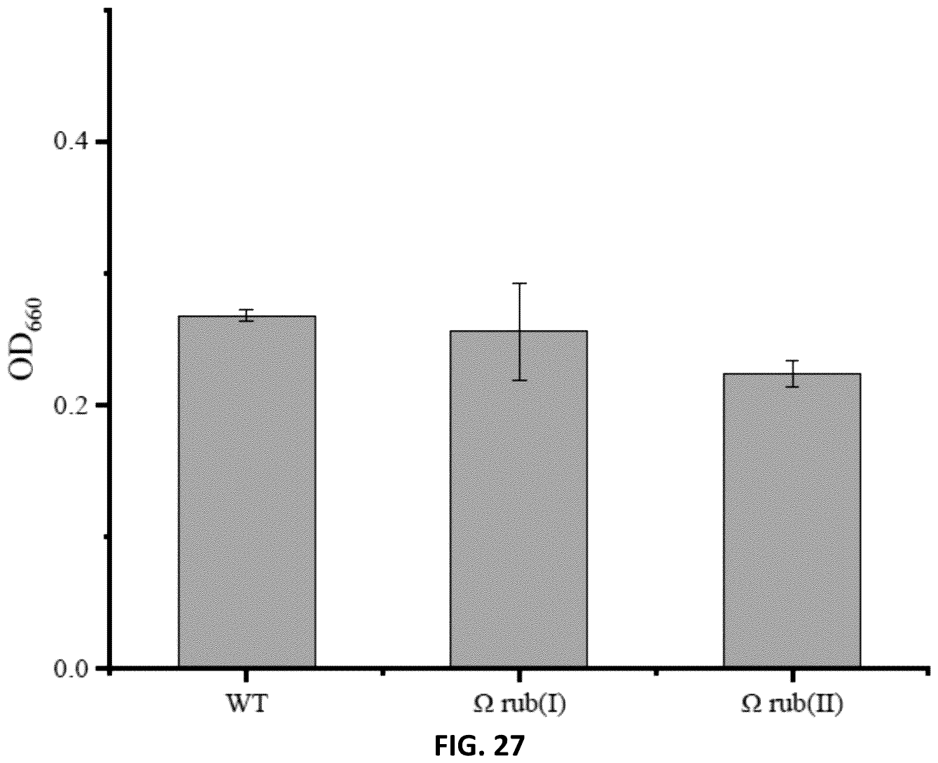

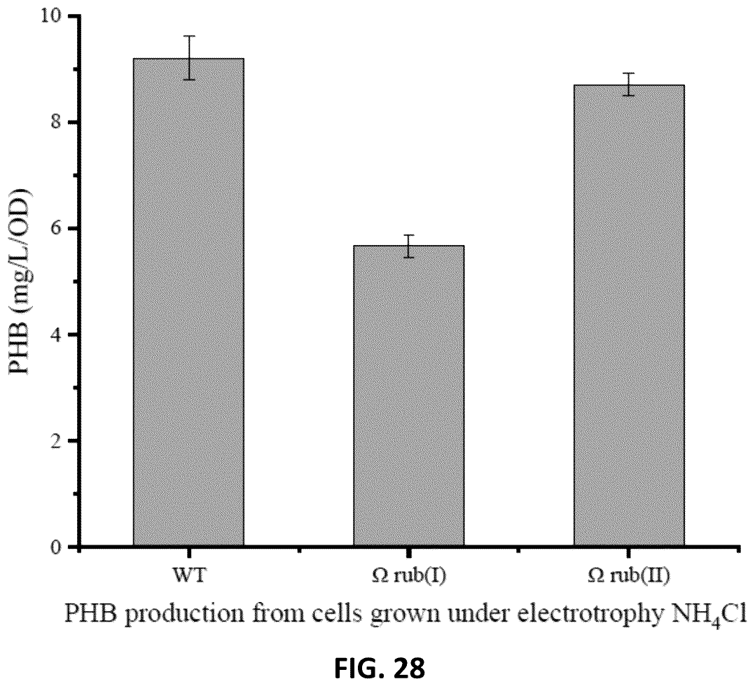

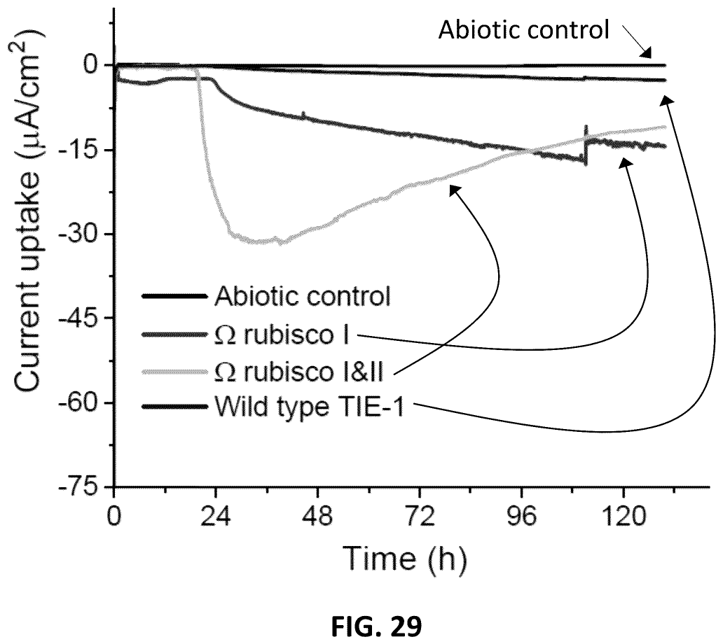

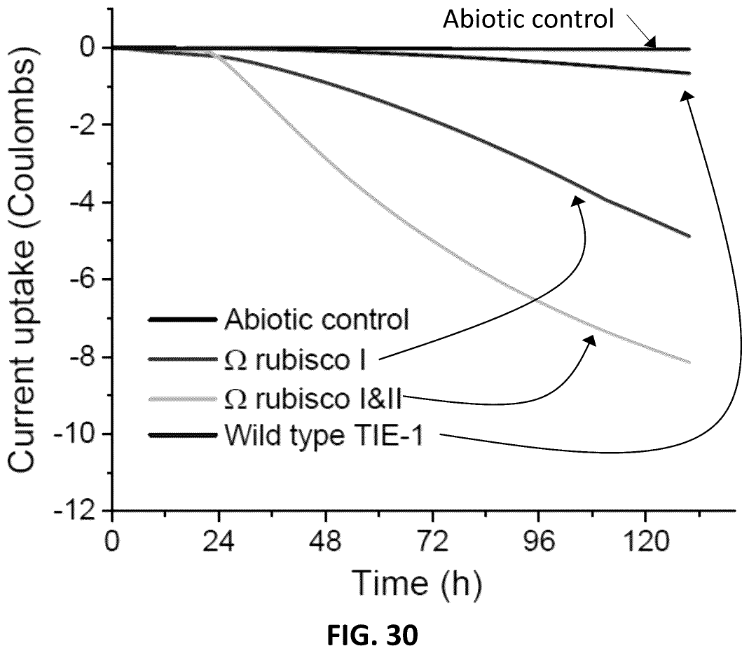

Strains were constructed using markerless integration. pWB107 and pWB108 were individually conjugated into TIE-1 through E. coli S17-1/A. After two successive homologous recombination, successful integrants were screened by PCR as shown in FIG. 19.

### TIE-1 Electroporation

To prepare electrocompetent cells, the TIE-1 strain was inoculated in 500 mL YPSMOPS and then incubated at 30° C. After reaching an OD660 of 0.5˜0.6, the culture was centrifuged and washed at 4° C. at 4000×g for 10 minutes. After five washes with 10% glycerol, the cell pellet was resuspended in 2 mL of ice-cold 10% glycerol. This resuspension was aliquoted by 50 μL per sample and then saved in −80° C. For every electroporation, 0.2 μg of plasmid was added to 50 μL thawed electrocompetent cells and mixed well. This mixture was added to 1 mm gap electroporation cuvettes and then cells electroporated at 1.8 kV using a Biorad gene Micropulser. After electroporation the mixture was added to 2 mL warmed Super Optimal Broth (SOB) or LB and grown for 45 minutes. 10 μL, 100 μL, 500 μL, and the remainder of the culture were plated on selective medium. Because these plates were further imaged by Nikon A1, glass Petri dishes instead of plastic Petri dishes were used.

Imaging of mCherry

After 4 to 5 days of incubation, the plated electroporated cultures expressing mCherry were imaged using the Nikon A1 confocal Eclipse Ti2 Microscope. For each plate, an image of the whole plate was captured using a camera through both the bright field and the Texas-red channel (excitation 586 nm, emission 603 nm) with a 10× objective. The colony number of each plate was quantified using NIS-Elements AR Analysis 5.11.01 64-bit software.

### Calculations of Transformation Efficiency and Colony Forming Unit per Optical Density

The φC31 phage integration systems were evaluated by calculating the transformation efficiency as calculated by the following equation:

$$Transformation efficiency = \frac{Colony number post - electroporation}{amount of plasmid ( μ g )}$$

The editing efficiency is calculated by:

$$Editing efficiency = \frac{Number of engineered colonies}{Number of initial colonies}$$

### Construction of TIE-1 Mutants

The following strains were constructed using the markerless deletion method as illustrated in FIG. 12: phaZ mutant 66 Rpal_0578, and phaR mutant (Δrpal_0531). The two mutants Δgly and the ΔnifA double mutant have been described previously. The mutant constructions were as follows: 1 kb upstream and 1 kb downstream of each of the genes of interest were PCR amplified and cloned into the cloning vector pJQ200KS. The constructed plasmids were then electroporated into the donor strain E. coli S17-1/A. The E. coli donor strains carrying the constructed plasmids were conjugated with the TIE-1 wild type. After two consecutive homologous recombination events, successful mutant candidates were screened using PCR (FIG. 13) and the primers listed in FIG. 9.

### PHA Extraction and Analysis

PHA samples were extracted from 5 mL of liquid bacterial culture and the organic phases were analyzed using LC-MS. 325 μL of methanol, 400 μL chloroform, and 75 μL of sulfuric acid were simultaneously added to the sample pellet. The mixtures were incubated at 95° C. for only one hour to minimize the potential loss of methylated products in the aqueous phase. Subsequently, phase separation and acid elimination were performed by the addition of 500 μL of LC-MS grade water. The organic phases were dried using a speed vacuum. Dried crotonic acid was then resuspended in 50% acetonitrile/50% water and analyzed using an Agilent Technologies 6420 Triple Quad LC/MS. Crotonic acid was detected with a mass mass-to-charge ratio (m/z) of 87. Standard curves of 1, 10, 50, and 100 ppm concentrations were created from a polyhydroxybutyrate (PHB) standards purchased from Sigma Aldrich. Standards were prepared the same way as the samples and were used for the determination of PHA from different samples.

PHA (mg/L) concentrations were determined using the standard curve generated from the counts obtained from the following dry powder PHB concentrations: 1, 10, 50 and 100 ppm.

PHA (mg/L/Cell) was obtained from the following equation:

$$PHA ( mg / L / Cell ) = \frac{PHA ( mg / L )}{Cell number}$$

PHA productivity in (mg/L/cell/hour) were obtained from the following equation:

$$PHA productivity = \frac{\frac{PHA}{cell number}}{growth time}$$

### OD Measurements, Cell Counts, and Cell Volume Determinations:

OD measurements were performed using a Spectronic 200 (Thermo Fisher Scientific, USA) at 660 nm. Cell enumeration was performed. Cell volume was determined by staining TIE-1 with FM 1-43FX membrane stain and visualized under a Nikon, A1 confocal microscope Model Eclipse Ti2. Stained cells were 3-D imaged using FM 1-43 laser channel at 471 nm. Cells were stained according to the manufacturer's recommendations with a slight modification. Briefly, 1 mL of culture cell of OD660˜1 was spun down at 15k rpm for one minute, washed once with 1×PBS, and resuspended in 100 μL of ice-cold methanol for 2 minutes. An additional wash with 100 μL ice-cold acetone was performed. Cells were finally resuspended in 1.5 mL 1×PBS buffer and stained with the FM-1-43FX dye for 10 minutes in the dark. Cells were mounted on a coverslip coated with 10% poly-L-lysine and air-dried before observation under the microscope. TIE-1 volume was determined as 1.048 pm³ using ˜1000 individual cells. PHA cell % v/v was obtained by dividing the volume of PHA per cell with the cell volume assuming that PHA density is 1.22 and the cell % w/w fresh weight was obtained by dividing PHA mass by the fresh cell mass assuming that the cell density is 1.1.

### Discussion

In Example 1, it is shown that TIE-1 can be modified using different genetic engineering and synthetic biology tools to improve PHA production. Deleting competitive pathways and regulating genes in the PHA cycle increased its production. Furthermore, overexpression of RuBisCo forms I and II using φC31 integrase in TIE-1 enhanced PHA production. These results show that the developed φC31 integration system is effective and useful for genetic manipulation in TIE-1.

### Integration System in TIE-1

Four φC31 expression systems were explored: inducible plasmid-based, constitutive genome-based (although not viable), and constitutive plasmid-based for φC31 integrase expression to determine the optimal integration system. It was observed that the inducible genome-based system proved to be most efficient. However, incorporating φC31 integrase into genomes of organisms poses significant challenges. Thus, using plasmid-based systems are significantly more straightforward. Between the two plasmid-based systems, using a constitutive promoter to drive φC31 integrase expression resulted in higher efficiency and did not leave the suicide plasmid behind. Thus, the plasmid-based system with the constitutive promoter stands out as an easy-to-use and efficient approach for integration using φC31 integrase in TIE-1.

Expression of PHA Production from Different TIE-1 Strains

Here, PHA is reported as % v/v, % 501 PHA (dry mass)/cell mass (fresh), mg/L/cell, and mg/L/cell/hour to express production (FIG. 11). Cell volume was estimated using microscopy techniques (as described in the Materials and Methods section) which enabled the use of PHA % v/v from individual cells. This cell volume was used to estimate PHA % dry PHA weight/cell fresh weight.

Effect of the Deletion of Genes from TIE-1 on its Growth and PHA Production

An increase in PHA production from ΔphaR was observed when grown with butyrate under non-nitrogen fixing conditions. However, switching to nitrogen-fixation caused a decrease in PHA production by ΔphaR. Deletion of the phaR resulted in a growth defect in TIE-1 both under photoheterotrophic with butyrate and photoautotrophic with H₂. It also decreased TIE-1's ability to oxidize Fe(II), and uptake electrons directly from an electrode especially under nitrogen-fixing conditions compared to wild type. Inactivation of the phaR in Bradyrhizobium diazoefficiens USDA110 not only decreased PHA accumulation but also affected other processes such as exopolysaccharide (EPS) biosynthesis, and heat stress tolerance. This suggests that the role of PhaR in Bradyrhizobium diazoefficiens USDA110 is not limited to PHA biosynthesis.

It was observed that deletion of phaZ did not significantly increase PHA production in TIE-1 (FIG. 10). However, deletion of phaZ affected the growth of TIE-1 under photoheterotrophic growth with butyrate regardless of the nitrogen source. A defect was also observed in the ability of ΔphaZ to oxidize Fe(II) during photoferrotrophic growth regardless of the nitrogen source. In addition, a decrease in electron uptake from ΔphaZ under nitrogen-fixing photoelectrotrophic growth conditions was also observed.

Deletion of the glycogen synthase (gly) gene did not affect the growth of TIE-1 under any conditions tested. However, a decrease in PHA production was observed under photoheterotrophic growth with butyrate and photoautotrophic growth under nitrogen-fixing conditions. This is similar to what was discovered previously in Synechocystis sp. PCC 6803, where glycogen was hypothesized to be a carbon source for PHA synthesis under nitrogen starvation. In contrast, PHA production increased under non-nitrogen fixing photoautotrophic growth conditions with H₂ in this mutant. This suggests that during photoautotrophy with fixed nitrogen present, elimination of glycogen accumulation allows carbon to be channeled toward increasing PHA reserves.

Effect of nitrogen fixation and deletion of the nifA genes on PHA production and growth of TIE-1: Nitrogen fixation has been previously reported to increase PHA production. This phenomenon was observed under photoheterotrophic growth conditions with butyrate as well as under photoautotrophic growth with H₂, mostly across all the strains. However, under photoautotrophic growth conditions with Fe(II), a decrease in PHA production was observed under nitrogen fixing compared to the non-nitrogen fixing conditions, whereas overall similar values were obtained from the photoelectrotrophic conditions under the two nitrogen sources. These observations align with previous observations. Once again, behavior was observed between the two groups of growth conditions: photoheterotrophic with butyrate and photoautotrophic growth with H₂ versus photoautotrophy with Fe(II) or poised electrodes.

Deletion of the nifA genes decreased PHA production under photoheterotrophic growth with butyrate under non-nitrogen fixing conditions which could be attributed to stress caused by the oxidation state of butyrate. However, similar to the increase in n-butanol production observed in ΔnifA, an increase in PHA production from this strain grown under non-nitrogen fixing photoautotrophic conditions with H₂ was noticed. Interestingly, an increase in PHA production was not observed by ΔnifA on any other growth conditions assessed. It was also observed that while all the mutant strains showed decreased PHA production under photoautotrophic growth with Fe(II) under non-nitrogen fixing conditions, ΔnifA's PHA production remained relatively close to wild type (FIGS. 10 and 5E).

### Effect of Overexpressing RuBisCo Genes on TIE-1's Growth and PHA Production

An increase of PHA production was observed in TIE-1 overexpressing RuBisCo form I and form I&II under photoheterotrophic with butyrate, photoautotrophic with H₂ and photoelectrotrophy under nitrogen-fixing conditions (FIGS. 10, 3F, 4F and 6E). An increase in carbon fixation by the CBB cycle is expected to increase the abundance of acetyl-CoA, the precursor of PHA which then leads to an increased PHA production in these strains.

A similar observation was reported from an overexpression of the RuBisCo gene in Ralstonia eutropha (now C. necator) grown autotrophically with minimal media (99.7% increase in PHA accumulation). ˜2× increase in overall PHA production (mg/L/Cell and cell % v/v) with the Ωrub(I) both was observed under photoheterotrophic with butyrate (both under non-nitrogen and nitrogen fixing conditions) and photoautotrophic with H₂ under non-nitrogen fixing conditions compared to wild type. Ωrub(I&II) demonstrated significantly higher PHA production, with ˜5× increase under photoheterotrophic growth with butyrate under non-nitrogen fixing, and ˜3× increase with butyrate under nitrogen-fixing conditions compared to wild type. A cumulative effect of overexpression of both RuBisCo form I and form II in increasing PHA production under photoheterotrophic growth with butyrate was also noticed. An increase of 2× in PHA production was observed in the Ωrub(I&II) under photoautotrophy with H₂ during non-nitrogen fixing conditions, and φrub(I) and Ωrub(I&II) under nitrogen-fixing photoelectrotrophic growth conditions. Surprisingly, Ωrub(I) showed a growth defect when grown photoheterotrophically with butyrate. Ωrub(I) also had a defect in its ability to oxidize Fe(II) when grown photoferrotrophically with Fe(II) under non-nitrogen fixing conditions. This defect in Fe(II) oxidation might be linked to the decrease in PHA production in Ωrub(I) under photoferrotrophy. The defect in PHA production in Ωrub(I) seems to disappear when both RuBisCo forms I and II (Ωrub(I&II) are overexpressed in TIE-1 regardless of the nitrogen source under growth with Fe(II). This suggests a compensatory effect of expressing both forms of RuBisCo on PHA production.

### Effect of the Deletion and Overexpression of Genes on Current Uptake During Photoelectrotrophic Growth of the Strains.

The difference in current uptake was more noticeable when the strains were grown under nitrogen-fixing conditions. The drastic decrease in current uptake observed from ΔphaR was reflected in the lower PHA produced unlike the rest of the strains (mutants and engineered strains) (FIGS. 6F and 11). The reason for the decrease in current uptake, alongside the increase in PHA production in the phaZ and gly mutants compared to wild type, is unclear as lower PHA production was anticipated by these mutants. Perhaps metabolic changes in these mutants redirects more intermediates to PHA biosynthesis. Additional investigation is required to clarify the connection between electron uptake and PHA production in these mutants. The RuBisCo overexpression strains showed both an increase in PHA production and current uptake under nitrogen-fixing conditions, which aligns with expectations.

It is concluded that overexpressing RuBisCo form I and II using the φC31 system increased PHA production in TIE-1. It would be of great interest to further investigate the role of PhaR as an increase in PHA production was observed under photoheterotrophic growth with butyrate under non-nitrogen fixing conditions in this study. Deletion of the glycogen synthase (gly) or nifA regulators increases PHA production under non-nitrogen fixing photoautotrophic growth with H₂. It would be interesting to assess the effect of creating combinatorial mutants of phaR, phaZ, gly, and the nifA regulator genes with simultaneous overexpression of RuBisCo form I and II genes (native and non-native) in TIE-1. The success of overexpression of the RuBisCo genes in increasing PHA production observed in this Example suggests that increasing expression of these genes above wild-type levels is valuable for bioproduction in autotrophic microbes.

### Example 2

### Improving the Polyhydroxybutyrate (PHB) Production in Rhodopseudomonas Palustris TIE-1

To tackle the environmental issues raised by the use of conventional plastic, during the past few years, this Example seeks to produce an alternative to conventional plastic. Using microorganisms that can produce bioplastic, bioplastic is produced, Two categories of strains have been designed in order to improve PHB in Rhodopseudomonas palustris TIE-1:1) mutants lacking genes and 2) engineered strains with overexpressing genes.
a) Mutants: i. lacking the glycogen gene: ΔRpal_(Rpal_0386). Being a carbon storage, deletion of the glycogen accumulation was created to remove the carbon competition towards PHB production.
ii. lacking the 2 nitrogenase regulator nifA1 and nifA2: Rpal_1624 and Rpal_5113 TIE-1 can fix nitrogen via its nitrogenase genes. Mutant lacking the nif genes have more reduced intracellular environment which is favorable for PHB production. iii. lacking the regulator phaR (Rpal_0531) of the PHB gene.

Located at the beginning of the PHB operon, this regulator gene might play an important role in the PHB pathway, deleting this regulator will certainly has an impact on the PHB cycle.
iii. Mutant lacking the PHB depolymerase phaZ (Rpal_0568). By deleting the depolymerase gene, the ability of the TIE-1 to degrade the polymer back into a carbon source could be altered. This mutant could potentially accumulate more PHB intracellularly
b) Engineered TIE-1 that have overexpressing RuBisCo from I and II integrated in the genome. Two strains of TIE-1 have been developed to increase its PHB production: TIE-1 overexpressing the RuBisCo form I (ΩrubI) the RuBisCo form I and II (ΩrubI&II) TIE-1 is a photoautotroph bacterium that has two ribulose bisphosphate carboxylase (RuBisCo) genes: Rpal_1747 and Rpal_5122 on its chromosome. During photosynthesis, RuBisCo helps fixing CO₂ in the Calvin Cycle which in turn gets transformed into glucose. Overexpression of these two genes were made possible by integrating them into the genome of TIE-1 and driven by a constitutively strong promoter PaphII using a φC31 integrase system.

Previously reported PHB production using TIE-1 includes two main categories of growth conditions, autotrophy and heterotrophy, which were tested using the mutants and the engineered strains developed in this project. In addition, studies under growth with NH₄Cl or under nitrogen fixing with N₂ were performed. the conditions tested are. butyrate, hydrogen, ferroautotrophy, and photoelectrotrophy.

The growth under hydrogen suggests that the at least under the growth with NH₄Cl, the engineered strain overexpressing the rub(I&II) is producing more PHB than the wild type.

Both engineered strains showed more electron uptake compared to the wild-type However, there is less PHB production from the Ωrub(I) compared to the wild type and the Ωrub(I&II) Overall, the results might suggest that the engineered strains Ωrub(I) and Ωrub(II).

What is claimed is:

1. A transgenic R. palustris TIE-1 microorganism comprising:
an exogenous RuBisCo ΩrubI gene; and
an exogenous RuBisCo ΩrubII gene.

2. The transgenic R. palustris TIE-1 microorganism of claim 1, further comprising an endogenous gene phaR deletion, such that the transgenic R. palustris TIE-1 microorganism lacks a functional copy of a phaR gene.

3. The transgenic R. palustris TIE-1 microorganism of claim 1, further comprising an endogenous gene phaZ deletion, such that the transgenic R. palustris TIE-1 microorganism lacks a functional copy of a phaZ gene.

4. The transgenic R. palustris TIE-1 microorganism of claim 1, further comprising:
an endogenous gene phaR deletion, such that the transgenic R. palustris TIE-1 microorganism lacks a functional copy of a phaR gene; and
an endogenous gene phaZ deletion, such that the transgenic R. palustris TIE-1 microorganism lacks a functional copy of: a phaR gene and a phaZ gene.

5. The transgenic R. palustris TIE-1 microorganism of claim 1, further comprising an endogenous gene gly deletion, such that the transgenic R. palustris TIE-1 microorganism lacks a functional copy of a gly gene.

6. The transgenic R. palustris TIE-1 microorganism of claim 1, further comprising an endogenous gene nifA deletion, such that the transgenic R. palustris TIE-1 microorganism lacks a functional copy of a nifA gene.

7. The transgenic R. palustris TIE-1 microorganism of claim 1, wherein the endogenous gene nifA deletion comprises deletion of a nifA1 gene and a nifA2 gene.

8. The transgenic R. palustris TIE-1 microorganism of claim 1, wherein the exogenous RuBisCo ΩrubI and ΩrubII genes are integrated into a genome of the transgenic R. palustris TIE-1 microorganism using a φC31 integrase system.

9. The transgenic R. palustris microorganism of claim 1, wherein the transgenic R. palustris microorganism is capable of increased bioplastic production compared to a wild-type R. palustris microorganism.

10. A method of producing polyhydroxybutyrate (PHB), the method comprising:
culturing a transgenic R. palustris TIE-1 microorganism in a culture medium, wherein the transgenic R. palustris TIE-1 microorganism comprises:
an exogenous RuBisCo ΩrubI gene; and
an exogenous RuBisCo ΩrubII gene; such that the exogenous RuBisCo ΩrubI and ΩrubII genes are overexpressed; and
recovering the PHB from the culture medium.

11. The method of claim 10, wherein the exogenous RuBisCo ΩrubI and ΩrubII genes are integrated into a genome of the transgenic R. palustris TIE-1 microorganism using a φC31 integrase system.

12. The method of claim 10, wherein the culture medium comprises photoheterotrophic conditions with butyrate and ammonium chloride.

13. The method of claim 10, wherein the culture medium comprises photoautotrophic conditions with H2 and ammonium chloride.

14. The method of claim 10, wherein the culture medium comprises photoelectrotrophic conditions with nitrogen.

15. A method of producing polyhydroxybutyrate (PHB), the method comprising:
culturing a transgenic R. palustris TIE-1 microorganism in a culture medium, wherein the transgenic R. palustris TIE-1 microorganism comprises:
an exogenous RuBisCo ΩrubI gene and an exogenous RuBisCo ΩrubII gene, such that the exogenous RuBisCo ΩrubI and ΩrubII genes are overexpressed;
an endogenous gene phaR deletion, such that the transgenic R. palustris TIE-1 microorganism lacks a functional copy of a phaR gene; and
an endogenous gene phaZ deletion, such that the transgenic R. palustris TIE-1 microorganism lacks a functional copy of a phaZ gene; and
recovering the PHB from the culture medium.

16. The method claim 15, wherein the exogenous RuBisCo ΩrubI and ΩrubII genes are integrated into a genome of the transgenic R. palustris TIE-1 microorganism using a φC31 integrase system.

17. The method of claim 15, wherein the culture medium comprises photoheterotrophic conditions with butyrate and nitrogen.

18. The method of claim 15, wherein the culture medium comprises photoheterotrophic conditions with butyrate and ammonium chloride.

19. The method of claim 15, wherein the culture medium comprises photoautotrophic conditions with hydrogen and ammonium chloride.

20. The method of claim 15, wherein the culture medium comprises photoelectrotrophic conditions with nitrogen.
